(19) United States
(12) Patent Application Publication
(10) Pub. No.: US 2025/0283119 A1
(43) Pub. Date: Sep. 11, 2025

(54) FERMENTATION PROCESSES AND SYSTEMS

(71) Applicant: MANUS BIO INC., Waltham, MA (US)

(72) Inventors: Carlos Rafael Castillo SALDARRIAGA, Stuggart (DE); Ralf TAKORS, Stuggart (DE); Ajikumar PARAYIL KUMARAN, Waltham, MA (US); Christine Nicole S. SANTOS, Waltham, MA (US)

(21) Appl. No.: 18/859,382

(22) PCT Filed: May 1, 2023
(86) PCT No.: PCT/US2023/020585

§ 371 Date: Oct. 23, 2024

Related U.S. Application Data
(60) Provisional application No. 63/336,320, filed on Apr. 29, 2022.

(51) Int. Cl. C12P 5/00; C12M 1/00; C12P 39/00; C12R 1/19

(52) U.S. Cl. CPC C12P 5/002; C12M 47/02; C12P 39/00; C12R 2001/19

(57) ABSTRACT

In various aspects and embodiments, this disclosure provides methods and systems for making a chemical product by microbial fermentation. In embodiments, the method comprises providing a cell culture producing the chemical product from carbon substrate or by enzymatic bioconversion of a fed substrate. In some aspects and embodiments, the cells are cultured in a fermentation media in a bioreactor under conditions suitable for producing the chemical product, while continuously harvesting filtered fermentation media from the bioreactor and feeding the culture with fresh media while retaining cultured biomass. The chemical product is recovered from the filtered fermentation media and/or the retentate.

### PRIORITY

This application claims the benefit of, and priority to, U.S. Provisional Application No. 63/336,320, filed Apr. 29, 2022, which is hereby incorporated by reference in its entirety.

### BACKGROUND

Fed-batch fermentation processes and systems are widely used. However, fed-batch processes have disadvantages such as an accumulation of toxic compounds, physical limitations due to volumetric reactor capacity, and complex control strategies to keep optimal conditions independently of volume changes. The present disclosure provides, among other things, continuous and semi-continuous fermentation systems based on biomass retention to overcome limitations of the fed-batch approach.

### SUMMARY OF THE DISCLOSURE

In various aspects and embodiments, this disclosure provides a method for making a chemical product by microbial fermentation. The method comprises providing a cell culture producing the chemical product from carbon substrate or by enzymatic bioconversion of a fed substrate. The cells are cultured in a fermentation media in a bioreactor under conditions suitable for producing the chemical product, while continuously harvesting filtered fermentation media from the bioreactor and feeding the culture with fresh media while retaining cultured biomass. The chemical product is recovered from the filtered fermentation media and/or the retentate. The present disclosure applies tools such as cross flow filtration modules or similar devices that can be used in situ, i.e. inside the bioreactor, or ex situ, i.e. outside the bioreactor, for partially or completely retaining biomass or product or both. The present disclosure mitigates challenges of the fed-batch approach using this ‘biomass retention’ process in which biomass is partially or completely retained and media is replaced in a frequent and/or continuous manner. The biomass retention process described herein enables continuous or semi-continuous production, reducing nutritional limitation events and intermittent harvest.

In some embodiments, the chemical product is recovered from the filtered media, for example, in continuous fashion. In these embodiments, the filter used for biomass retention does not retain a substantial amount of the product. In some embodiments, the chemical product is recovered from the retentate. That is, the filter used for biomass retention also retains the product or a substantial amount of the product. In still other embodiments, the chemical product is recovered from the filtered media, as well as the retentate, for example, in embodiments where the product is significantly retained with the biomass as well as present in the filtered broth.

In various embodiments, including for semi-continuous processes involving intermittent harvest of the product from the retentate, the productive phase is maintained for at least about 72 hours, but can be 336 hours or more. In various embodiments, the full volume of media in the bioreactor is replaced at least twice during the production phase, or can be replaced twenty times or more during the production phase. In various embodiments, including where product is substantially retained by in the bioreactor by the filtration module, the volume of media in the bioreactor is replaced from two to ten times, or from four to ten times, during the production phase. In embodiments where the product is substantially present in filtered broth, the process can be essentially continuous, in which the volume of media is replaced more than 20 times, or more than 50 times.

In various embodiments, the process described herein allows for higher production of desired chemical products by fermentation at lower bioreactor volumes, as compared to conventional fed batch methods.

In some embodiments, the bioreactor comprises a filtration probe or an external filtration module filtering fermentation media from biomass. In some embodiments, the filtration probe or filtration module comprises a hydrophobic or hydrophilic membrane filtering the fermentation media from biomass. In various embodiments, the filter is approximately a 0.2 micron filter, to restrict passage of cells/biomass. In embodiments, the filtration probe or module is operated by a pump to control the rate by which fermentation broth is removed from the bioreactor.

In various embodiments, fermentation media is extracted in situ (i.e., within the bioreactor) using a filtration probe. The filtration probe can be a tubular microfiltration device. Generally, the filtration probe will be constructed from a polymeric material (including but not limited to polypropylene as demonstrated herein). In some embodiments, the desired chemical product is retained by the filter (e.g., the filter material is selected or designed to retain the chemical product along with cells), thereby concentrating the chemical product in the retentate. In certain embodiments, the bioreactor is operably connected to an external filtration module (ex situ), which can allow for cross flow filtration or filtration by another means. In some embodiments, a plurality of bioreactors are operably connected to one or more external filtration modules for ex situ filtration. In some embodiments, filtered broth is collected for recovery of the desired chemical product, and which in some embodiments can be performed continuously.

Retention of the desired chemical product can facilitate downstream processing, given the low volume of liquid and high concentration of the desired chemical product. In such embodiments, the disclosure provides a semi-continuous process that can employ batch harvesting after several cycles of biomass retention.

In various embodiments, the cell (e.g., of the cell culture) is a bacterium, such as but not limited to E. coli. In some embodiments, the process employs a bacterium (such as E. coli) having a modified genome to facilitate large scale processes. Thus, the process can employ a genome-modified bacterial strain that reduces energetically wasteful cellular responses during large-scale culture by deletion or inactivation of non-essential genes.

In various embodiments, the microorganism (e.g., of the cell culture) is a yeast. In various embodiments, the yeast cell is selected from a species of Saccharomyces, Pichia, or Yarrowia, such as Saccharomyces cerevisiae, Pichia pastoris, and Yarrowia lipolytica.

In various embodiments, the chemical product is synthesized enzymatically by a recombinant biosynthetic pathway. In some embodiments, when using a consortium, the recombinant biosynthetic pathway is expressed across at least two or at least three or at least four microbial cell populations. According to embodiments of this disclosure, the chemical product may be a terpene, terpenoid, alkaloid, cannabinoid, steroid, saponin, glycoside, stilbenoid, polyphenol, flavonoid, antibiotic, polyketide, fatty acid (e.g., short chain, medium chain, or long chain), triglyceride, peptide, or recombinant protein. In some embodiments, the chemical product is a terpene or terpenoid, such as a monoterpene or monoterpenoid, a sesquiterepene or sesquiterpenoid, or a diterpene or diterpenoid.

In various embodiments, a microbial cell population expresses heterologous gene encoding one or more of a uridine diphosphate dependent glycosyltransferase enzyme (UGT), a methyltransferase enzyme, a acetyltransferase enzyme, or a benzoyl transferase enzyme. For example, in embodiments employing a UGT enzyme, the chemical product can be selected from a terpenoid glycoside, flavonoid glycoside, cannabinoid glycoside, polyketide glycoside, stilbenoid glycoside, and polyphenol glycoside. In some embodiments, the desired chemical product is produced by whole cell bioconversion of a fed substrate. Such bioconversion processes include glycosylations of a fed substrate (including but not limited to terpenoid glycosides such as steviol glycosides and mogrosides). In such embodiments, the fed substrate is a plant extract.

The biomass retention process described herein can employ bioreactors with mechanical agitation (e.g., a stirred tank reactor). Alternatively, the process can employ bioreactors that do not involve mechanical agitation, such as a bubble column reactor.

In other aspects, this disclosure provides a fermentation system for conducting the method disclosed herein. For example, in some embodiments employing an in situ filtration module, the invention provides a fermentation system comprising a bioreactor vessel containing growth media and a microbial biomass. The system further comprises: a filtration membrane removing permeate from the bioreactor vessel in situ while retaining biomass; a feeding vessel comprising fresh broth that is in fluid connection with the bioreactor vessel; and a vessel for collecting filtrated broth that is in fluid connection with the filtration membrane. In some embodiments employing an ex situ filtration module, the invention provides a fermentation system comprising a bioreactor vessel containing growth media and a microbial biomass. The system further comprises: a cross filtration module (CFM) fluidly connected to the bioreactor vessel, the CFM comprising a membrane removing permeate from the growth media ex situ while retaining biomass; a feeding vessel comprising fresh broth that is in fluid connection with the bioreactor vessel; and a vessel fluidly connected to the CFM for collecting filtrated broth, while retentate comprising biomass is recirculated to the bioreactor via a recirculation line.

In still other aspects, the disclosure provides methods for microbial fermentation that limit and/or avoid the use of yeast extract and other complex media for creating biomass and/or for the production phase, since yeast extract is costly and can complicate downstream processing. In these aspects, microbial strains are cultured in fed-batch, semi-continuous, or continuous systems with yeast extract partly or completely replaced with a defined media comprising an amino acid mix. In some embodiments, the defined media comprises supplementation with pyruvate.

Further aspects and embodiments of the invention will be apparent from the following detailed disclosure.

### FIGURES

FIG. 1A shows the relative dry cellular weight (DCW) during fermentation using embodiments of the presently disclosed biomass retention process as compared to a fed-batch process. The biomass retention process shows about a 40% increase in biomass DCW over the fed-batch process.

FIG. 1B shows relative AMD4,11 concentration during the fermentation course using an embodiment of the disclosed biomass retention process and fed batch. The biomass retention process resulted in over 80% improvement in product with extended production times.

FIG. 2A-C compare biomass specific productivity using the biomass retention process according to embodiments of the disclosure and a fed-batch process. FIG. 2A shows the relative final amorpha-4,11-diene production. FIG. 2B shows the relative AMD4,11 production rate between fed-batch and the biomass retention strategy, and FIG. 2C shows AMD4,11 conversion yield between fed-batch and the biomass retention strategy.

FIG. 3A-C show the incremental improvements in AMD4,11 production using a single cycle or repetitive biomass retention processes of the present disclosure as compared to a fed batch process. FIG. 3A shows total AMD4,11 produced. FIG. 3B shows reactor productivity. FIG. 3C shows reductions in time using the repetitive biomass retention approach of the instant disclosure, as compared to fed-batch.

FIG. 4A-D illustrates AMD4,11 productivity using a biomass retention approach according to this disclosure. FIGS. 4A and 4B shows fold change in AMD4,11 production across 4 cycles of media exchange in a repetitive biomass retention process. FIG. 4C shows distribution of AMD4,11 produced per cycle in a 3 and 4 cycle process. FIG. 4D shows AMD4,11 conversion yield across 4 cycles of media exchange in a repetitive biomass retention process.

FIG. 5A shows dry cellular weight (g/L) during a fed-batch fermentation with pyruvate supplementation (T1=0.09 g/h; T2=0.18 g/h; T3=0.27 g/h) with respect to a control treatment at different sampling point (48, 60, 72 h). FIG. 5B shows total amount of AMD4,11 produced during a fed-batch fermentation with pyruvate supplementation (T1=0.09 g/h; T2=0.18 g/h; T3=0.27 g/h) with respect to a control treatment.

FIG. 6A shows batch productivity (mg/h) during a fed-batch fermentation with pyruvate supplementation (T1=g/h; T2=0.18 g/h; and T3=0.27 g/h) with respect to a control treatment in two time frames, up to 52 h and 60-72 h. FIG. 6B shows conversion yield (mg AMD4,11/g glucose) during a fed-batch fermentation with pyruvate supplementation (T1=0.09 g/h; T2=0.18 g/h; T3=0.27 g/h) with respect to a control treatment in three time frames: 0-12 h (batch phase), 12-52 h (fed-batch without supplementation), and 60-72 h (fed-batch with supplementation of pyruvate).

FIG. 7A shows the total amount of AMD4,11 produced during a repetitive biomass retention (RBR) process using different media composition (n=2). Media compositions are summarized in Table 1. FIG. 7B shows conversion yield to AMD4,11 during a repetitive biomass retention process using the different media composition (n=2).

FIG. 8 shows AMD4,11 batch productivity during a repetitive biomass retention process using different media composition (n=2).

FIG. 9 shows cell dry weight concentration normalized by the maximum achieved value in a biomass retention process by multiple cycles with the strain DMD6 (producing AA).

FIG. 10A shows DHAA and AA concentration normalized by the maximum achieved value in a biomass retention process by multiple cycles with the strain DMD6.

FIG. 10B shows DHAA and AA concentration in the filtered broth normalized by the mean value (n=3). Both DHAA and AA were detected in the filtered broth from the filtration module.

FIG. 11 illustrates a stirred tank reactor design (mechanical agitation) with in situ extraction of fermentation media.

FIG. 12 illustrates a stirred tank reactor design (mechanical agitation) and an external (ex situ) extraction module.

FIG. 13 illustrates a system employing multiple stirred tank reactors (mechanical agitation) connected to an external (ex situ) extraction module.

FIG. 14 illustrates a bubble column reactor (non-mechanically agitated) with in situ extraction of fermentation media.

FIG. 16 illustrates a bubble column reactor (non-mechanically agitated) with an external (ex situ) extraction module.

### DETAILED DESCRIPTION OF EMBODIMENTS

In various aspects and embodiments, this disclosure provides a method for making a chemical product by microbial fermentation. The method comprises providing a cell culture producing the chemical product from carbon substrate or by enzymatic bioconversion of a fed substrate. In some embodiments, the cell culture produces the chemical product through one or more heterologously expressed enzymes. The cells are cultured in a fermentation media in a bioreactor under conditions suitable for producing the chemical product, while continuously harvesting filtered fermentation media from the bioreactor and feeding the culture with fresh media while retaining cultured biomass. The chemical product is recovered from the filtered fermentation media and/or the retentate.

The present disclosure makes use of complete or partial retention of cells, and optionally retention of product, as described herein. The present disclosure applies tools such as cross flow filtration modules or similar devices that can be used in situ, i.e. inside the bioreactor, or ex situ, i.e. outside the bioreactor, for partially or completely retaining biomass or product or both. The present disclosure demonstrates the continuous/semi-continuous systems in the production of amorpha-4,11-diene (AMD4,11), which is a sesquiterpene used as a precursor of artemisinin, a potential antimalarial drug. The disclosure further demonstrates the process for the production of dihydroartemisinic acid (DHAA) and artemisinic acid (AA), also precursors of artemisinin. Conventionally, sesquiterpenes and other fermentation products produced using platforms such as Escherichia coli employ fed-batch processes that apply nutritional limiting conditions to reach a non-growth associated production stage. The present disclosure mitigates challenges of the fed-batch approach using a so-called ‘biomass retention’ process in which biomass is partially or completely retained and media is replaced in a frequent and/or continuous manner. In various embodiments of this disclosure, the specific production rate and conversion yield is kept constant at high cell densities. Furthermore, the biomass retention process described herein enables continuous or semi-continuous production, reducing nutritional limitation events and intermittent harvest.

This disclosure demonstrates the invention by evaluating the application of a biomass retention process by single and multiple cycles in the production of the sesquiterpene AMD4,11, DHAA, and AA, via the methylerythritol 4-phosphate pathway (MEP pathway) in E. coli. As used herein, the term “cycle” in association with a biomass retention approach refers to a duration of the production phase where 50% of the fermented broth is replaced with fresh medium. With a single biomass retention cycle under carbon limiting conditions, it was possible to increase the dry cellular weight by about 40% without reducing the AMD4,11-specific biomass production rate or conversion yield, which increased the product concentration up to about 80% and decreased the process time by about 26%, in comparison to a traditional fed-batch process using as a calculation basis the total amount of AMD4,11.

The processes described herein are applicable to other products, including other secondary metabolites (including natural compounds) and recombinant protein. The processes described herein are applicable to various microbial fermentation or whole cell bioconversion systems, including bacterial and yeast systems. In various embodiments, the invention can reduce process time associated with preparation activities (cleaning and sterilization) or inoculum production and increase reactor productivity due to the biomass concentration under optimal production conditions.

In some embodiments, the chemical product is recovered from the filtered media, for example, in continuous fashion. In these embodiments, the filter used for biomass retention does not retain a substantial amount of the product. In some embodiments, the chemical product is recovered from the retentate. That is, the filter used for biomass retention also retains the product or a substantial amount of the product. In still other embodiments, the chemical product is recovered from the filtered media, as well as the retentate, for example, in embodiments where the product is significantly retained and present in the filtered broth.

In various embodiments, the volume of the fermentation media in the bioreactor is kept substantially constant during the culturing. In various embodiments, the volume of the fermentation media does not vary by more than about 15%, or more than about 10%, or more than about 5%, during a production phase. As used herein, the “production phase” is the phase of culturing in which biomass is kept relatively constant while the cell culture produces the chemical product. The production phase generally involves carbon limiting conditions (e.g., comprising feeding a nitrogen source and a carbon source) and is distinguished from a growth phase, in which biomass is produced.

In some embodiments, the process comprises a first phase where microbial biomass is created, followed by a production phase involving biomass retention. In various embodiments, a base medium supports initial cell culture (i.e., for biomass production) and a feed medium is added to prevent nutrient depletion. The controlled addition of the nutrient directly affects the growth rate of the culture and helps to avoid overflow metabolism and formation of side metabolites. An exemplary base media can comprise, without limitation, yeast extract. In some embodiments, carbon substrates such C1, C2, C3, C4, C5, and/or C6 carbon substrates are fed to the culture during the production phase for production of the terpene or terpenoid product. In exemplary embodiments, the carbon source is glucose, sucrose, fructose, xylose, and/or glycerol.

Culture conditions are generally selected from aerobic, microaerobic, and anaerobic. In some embodiments, the biomass production phase takes place under aerobic conditions, followed by reducing the oxygen levels for the product production phase. For example, the culture can be shifted to microaerobic conditions after from about 10 to about 20 hours. In this context, the term “microaerobic conditions” means that cultures are maintained just below detectable dissolved oxygen. See, Partridge J D, et al., Transition of Escherichia coli from Aerobic to Micro-aerobic Conditions Involves Fast and Slow Reacting Regulatory Components, J. Biol. Chem. 282 (15): 11230-11237 (2007).

The production phase comprises feeding a nitrogen source and a carbon source. For example, the nitrogen source can comprise ammonium (e.g., ammonium hydroxide). The carbon source may contain C1, C2, C3, C4, C5, and/or C6 carbon sources, such as, in some embodiments, glucose or glycerol. The nitrogen and carbon feeding can be initiated when a predetermined amount of batch media is consumed. In various embodiments, the media used for the production phase, and which is continually or frequently fed to the system, further comprises base media such as yeast extract or other well-known base media. In some embodiments, the media for production phase comprises amino acids, which can replace yeast extract partially or completely. An exemplary amino acid mix (used herein for an E. coli culture) comprises, consists essentially of, or consists of L-Ala, L-Glu, Gly, L-Leu, L-Val, and L-Ile. Various amino acid mixes are known in the art and can be selected or tailored for the microbial species or strain. In some embodiments, amino acid mix is employed together with yeast extract. In some embodiments, amino acids are fed to the system during the production phase without yeast extract. In embodiments that avoid substantial use of yeast extract, downstream processing can be facilitated, in view of the complex nature of yeast extract.

In still other embodiments, the culture is supplemented with pyruvate during the production phase. In some embodiments, the culture media supplied during the production phase provides from about 1% to about 3% of carbon in the form of pyruvate (i.e., or salt thereof) (e.g., about 2% of carbon from pyruvate or salt thereof).

Any carbon source may be employed in connection with this disclosure, and the selection of carbon source may be selected based upon the cell culture as well as other factors such as cost. In various embodiments, the carbon source comprises one or more mono-, di-, or oligosaccharides, and in some embodiments may comprise more complex polysaccharides as well as various C1 to C6 carbon sources. Exemplary carbon sources include glucose, sucrose, fructose, xylose, and glycerol. In still other embodiments, the carbon source may comprise an organic stream selected from processed lignocellulosic biomass or municipal waste, or a carbon source derived therefrom. In some embodiments, the carbon source comprises CO₂ or biogas generated by anaerobic digestion (e.g., comprising methane).

In various embodiments, the production rate (i.e., during the production phase) of the chemical product is kept substantially constant. In some embodiments, the production rate does not vary by more than about 20%, or more than about 15%, or more than about 10% during the production phase.

In various embodiments, including for semi-continuous processes involving intermittent harvest of the product from the retentate, the productive phase is maintained for at least about 72 hours, or at least about 96 hours, or at least about 120 hours, or at least about 144 hours, or at least about 168 hours, or at least about 192 hours, or at least about 216 hours, or at least about 240 hours, or at least about 288 hours, or at least about 336 hours.

In various embodiments, the levels of media components and pH are kept substantially constant during the production phase (i.e., through the replacement of fresh media). In some embodiments, concentration of nitrogen is kept substantially constant during the production phase. During the production phase, the culture is maintained at high cell density, and in various embodiments, at least about 0.1 gram of dry cells per L, 1 gram of dry cells per L, or at least about 5 grams of dry cells per L, or at least about 8 grams of dry cells per L, or at least about 10 grams of dry cells per L, or at least about 12 grams of dry cells per L, or at least about 15 grams of dry cells per L, or at least about 20 grams of dry cells per L, or at least about 25 grams of dry cells per L, or at least about 30 grams of dry cells per L, or at least about 35 grams of dry cells per L, or at least about 40 grams of dry cells per L. The actual cell density will depend on the type of cell culture, including bacterial or yeast cultures as described herein.

In various embodiments, the full volume of media in the bioreactor is replaced at least twice during the production phase, or at least four times during the production phase, or at least six times during the production phase, or at least eight times during the production phase, or at least ten times during the production phase, or at least twenty times during the production phase. In various embodiments, including where product is substantially retained in the bioreactor by the filtration module, the volume of media in the bioreactor is replaced from two to ten times, or from four to ten times, during the production phase. In embodiments where the product is substantially present in filtered broth, the process can be essentially continuous, in which the volume of media is replaced more than 20 times or more than 50 times.

In various embodiments, during the production phase every 24 hours at least about 5% of the media volume is replaced, or at least about 10% of the media volume is replaced, or at least about 15% of the media volume is replaced, or at least about 20% of the media volume is replaced, or at least about 25% of the media volume is replaced, or at least about 30% of the media volume is replaced, or at least about 35% of the media volume is replaced, or at least about 40% of the media volume is replaced, or at least about 45% of the media volume is replaced, or at least about 50% of the media volume is replaced. In various embodiments, every 24 hours from 5% to about 50% of the media volume is replaced, or from about 10% to about 40% of the media volume is replaced, or from about 10% to about 30% of the media volume is replaced, or from about 15% to about 35% of the media volume is replaced, or about 25% of the media volume is replaced. In various embodiments, the media is not replaced during the growth phase.

In various embodiments, the process described herein allows for higher production of desired chemical products by fermentation at lower bioreactor volumes, as compared to conventional fed batch methods. In some embodiments, the volume of the media in the bioreactor is about 250,000 L or less, or about 150,000 L or less, or about 100,000 L or less, or is about 50,000 L or less, or is about 25,000 L or less, or is about 10,000 L or less, or is about 1000 L or less. In various embodiments, the volume of the media in the bioreactor is from about 10,000 L to about 250,000 L, or from about 10,000 L to about 100,000 L. In some embodiments, lower bioreactor volumes reduce the amount of time required for the growth phase.

In some embodiments, the bioreactor comprises a filtration probe or an external filtration module filtering fermentation media from biomass. In some embodiments, the filtration probe or filtration module comprises a hydrophobic or hydrophilic membrane filtering the fermentation media from biomass. In an exemplary embodiment, the filtration probe or module comprises a polypropylene membrane filtering the fermentation media from biomass. In embodiments, the filtration probe or module comprises a polyethersulfone (PES) filter, a nylon filter, cellulose acetate filter, or cellulose nitrate filter. In some embodiments, the filtration probe or module comprises a polyfluoroalkyl filter. In various embodiments, the filter is approximately a 0.2 micron filter, to restrict passage of cells/biomass. In embodiments, the filtration probe or module is operated by a pump.

In various embodiments, fermentation media is extracted in situ (i.e., within the bioreactor) using a filtration probe. The filtration probe can be a tubular microfiltration device. Generally, the filtration probe will be constructed from a polymeric material (including but not limited to polypropylene as demonstrated herein) and will contain pore sizes sufficient to restrict entry of whole cells (e.g., 0.2 microns). Other exemplary polymeric materials are described above. The surface area of the membrane can be adjusted according to the volume of the reactor and required flow of volume through the filtration probe. In certain embodiments, the bioreactor is operably connected to an external filtration module (ex situ), which can allow for cross flow filtration or filtration by another means. The filtration membrane and surface area can likewise be selected and controlled as described herein.

In some embodiments, a plurality of bioreactors are operably connected to one or more external filtration modules for ex situ filtration. In some embodiments, filtered broth is collected for recovery of the desired chemical product, and which in some embodiments can be performed continuously. In still other embodiments, the desired chemical product is retained by the filter (e.g., the filter material is selected or designed to retain the chemical product along with cells), thereby concentrating the chemical product in the retentate, as demonstrated herein for AMD using the disclosed hydrophilic membrane. Retention of the desired chemical product can facilitate downstream processing, given the low volume of liquid and high concentration of the desired chemical product. In such embodiments, the disclosure provides a semi-continuous process that can employ batch harvesting after several cycles of biomass retention (e.g., from 4 to 10 cycles). In accordance with certain embodiments of this disclosure, the fermentation system need not employ an organic overlayer to sequester the chemical product. However, in other embodiments, an organic overlayer can be fed to the system, e.g., for continuous or semi-continuous harvest of the chemical product. In some embodiments, the filter membrane can be designed to allow for an organic phase, such as the use of hydrophilic-hydrophobic hybrid polymers that allow for harvesting of organic overlayer. The composition of organic overlayers can be selected as described in U.S. 2021/0207078, which is hereby incorporated by reference in its entirety.

In various embodiments, the cell (e.g., of the cell culture) is a bacterium. In embodiments, the bacterium belongs to a genus selected from Acidovorax, Acinetobacter, Actinomyces, Alcanivorax, Arthrobacter, Brevibacterium, Bacillus, Clostridium, Corynebacterium, Deinococcus, Dietzia, Escherichia, Gordonia, Marinobacter, Mycobacterium, Micrococcus, Micromonospora, Moraxella, Nocardia, Pseudomonas, Psychrobacter, Rhodobacter, Rhodococcus, Salmonella, Streptomyces, Thalassolituus, Thermomonospora, Vibrio, and Zymomonas. In some embodiments, the bacterium is a species selected from Rhodococcus opacus, Acinetobacter calcoaceticus, Streptomyces coelicolor, Rhodococcus jostii, and Acinetobacter baylyi. In still other embodiments, the bacterium is a species of Escherichia, Bacillus, Corynebacterium, Deinococcus, Rhodobacter, Zymomonas, Pseudomonas, Vibrio, and Zymomonas. For example, the bacterium may be selected from Escherichia coli, Bacillus subtilis, Corynebacterium glutamicum, Rhodobacter capsulatus, Rhodobacter sphaeroides, Zymomonas mobilis, Pseudomonas putida, and Vibrio natriegens. In certain embodiments, the bacterium is E. coli, which can optionally employ the cell densities as already described.

In some embodiments, the process employs a bacterium (such as E. coli) having a modified genome to facilitate large scale processes, as described in WO 2022/094445, which is hereby incorporated by reference in its entirety. For example, in large-scale production processes bacterial cells are exposed to heterogeneous substrate availability caused by long mixing times. Bacterial cells, when moving transiently through nutrient poor zones, react by looping accumulation of the alarmone ppGpp and energetically wasteful cellular responses, resulting in growth and productivity limitations. Thus, in some embodiments, the process employs a genome-modified bacterial strain that reduces energetically wasteful cellular responses during large-scale culture by deletion or inactivation of non-essential genes. It is believed that the deletions avoid costly and unnecessary DNA replication, RNA synthesis, protein synthesis, and/or other ATP-intensive cellular processes during large scale culture. Such gene deletions or inactivations include those coding for flagella components or transcriptional regulators thereof, chemotaxis proteins and regulators thereof, inhibitors of DNA replication, and proteins involved in active transport of sugars other than glucose, among other cellular processes disclosed herein. According to these embodiments, such strains exhibit a lower cost of maintenance and higher biosynthesis performance under large-scale conditions as compared to a parent strain that does not contain said deletions or inactivations.

In various embodiments, the bacterial strain has deletions or inactivations of genes encoding flagella components or transcriptional regulators thereof. For example, in some embodiments, the bacterial strain is a strain of E. coli, and deleted or inactivated genes encoding flagella components or transcriptional regulators thereof are selected from: fliA, flk, fliC, flgA, flgB, flgC, flgD, flgE, flgG, flgH, flgI, flgJ, flgK, flgL, fliE, fliF, fliG, fliH, flil, fliJ, flik, fliL, fliM, fliN, fliO, flip, fliQ, fliR, flhE, flhA, and flhB. In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In various embodiments, one or more operons encoding flagella components are deleted, or operon expression is otherwise reduced or eliminated.

In these or other embodiments, the strain comprises deletions or inactivations of one or more genes encoding chemotaxis proteins and regulators thereof. For example, the strain may comprise deletions or inactivations of one or more of E. coli genes tar, tap, cheR, cheB, cheY, cheZ, cheW, and cheA. In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In various embodiments, one or more operons encoding such genes are deleted, or expression of the operon otherwise reduced or eliminated.

In some embodiments, the strain has a deletion or inactivation of one or more genes involved in active transport of sugars, especially sugars other than glucose. Exemplary genes according to these embodiments include one or more of E. coli gatA, gatB, gatC, gatD, gatR, and uhpT (or orthologs thereof). In various embodiments, one or more of such genes may be substantially deleted, or promoters deleted or inactivated, or necessary transcription factors deleted or inactivated (at the DNA or protein level), to thereby avoid costly DNA, RNA, and/or protein synthesis. In various embodiments, one or more operons encoding such genes are deleted, or expression of the operon otherwise reduced or eliminated.

Thus, in some embodiments, the bacterial strain comprises deletions or gene inactivations selected from: fliA, flk, fliC, flgA, flgB, flgC, flgD, flgE, flgG, flgH, flgI, flgJ, flgK, flgL, fliE, fliF, fliG, fliH, flil, fliJ, flik, fliL, fliM, fliN, fliO, flip, fliQ, fliR, flhE, flhA, flhB, cheZ, cheY, cheB, cheR, tap, tar, cheW, cheA, motB, motA, cspD, aldA, gatA, gatB, gatC, gatD, gatR, uhpT, yeeL, and flxA.

In various embodiments, the microorganism (e.g., of the cell culture) is a yeast. In some embodiments, the yeast is a species of Ashbya, Aspergillus, Aurantiochytrium, Bastobotyrs, Candida, Claviceps, Cryptococcus, Cunninghamella, Geotrichum, Hansenula, Issatchenkia, Kluyveromyces, Kodamaea, Leucosporidiella, Linderna, Lipomyces, Mortierella, Myxozyma, Mucor, Occultifur, Ogataea, Penicillium, Phaffia, Pichia, Prototheca, Rhizopus, Rhodosporidium, Rhodotorula, Saccharomyces, Scheffersomyces, Schizosaccharomyces, Sporidiobolus, Sporobolomyces, Starmerella, Tremella, Trichosporon, Wickerhamomyces, Waltomyces, or Yarrowia. In some embodiments, the yeast or fungal cell is a species selected from Yarrowia lipolytica, Yarrowia phangngensis, Pichia kudriavzevii, Saccharomyces cerevisiae, Pichia pastoris, Kluyveromyces marxianus, Rhodosporidium toruloides, Sporidiobolus ruinenii, Sporidiobolus salmonicolor, Aspergillus oryzae, Mortierella isabellina, Waltomyces lipofer, Candida tropicalis, Candida boidinii, Scheffersomyces stipitis, Mucor circinelloides, Ashbya gossypii, Trichoderma harzianum, Pichia guilliermondii, Kodamaea ohmeri, Rhodotorula aurantiaca, Lindnera saturnus, Penicillium roqueforti, Lipomyces starkeyi, and Bastobotyrs adeninivorans.

In various embodiments, the yeast cell is selected from a species of Saccharomyces, Pichia, or Yarrowia, such as Saccharomyces cerevisiae, Pichia pastoris, and Yarrowia lipolytica.

In some embodiments, the cell uses CO₂ as a carbon source, and may be an algae or cyanobacteria, such as Synechoccus elongatus, or acetogenic bacteria. In some embodiments, the algae is microalgae. In such embodiments, the bioreactor design allows the microbial cells to be exposed to sunlight.

In certain embodiments, the biomass retention process employs a microorganism consortium that produces the desired chemical product. See, for example, Wang R, et al., Recent advances in modular co-culture engineering for synthesis of natural products. Current Opinion in Biotechnology 2020, 62:65-71; Diender M, et al., Synthetic co-cultures: novel avenues for bio-based processes, Current Opinion in Biotechnology 2021, 67:72-79; and Xu P, et al., Microbial Coculture for Flavonoid Synthesis, Trends in Biotechnology, 2020. For example, the consortium comprises at least a first microbial cell population that converts a carbon source to an intermediary metabolite, and one or more additional microbial cell populations that convert the intermediary metabolite to the chemical product. As an example, the first microbial cell population may be a cyanobacteria such as Synechoccus elongatus, which produces sucrose from a CO₂ carbon source; and one or more additional microbial cell populations produce the chemical product from sucrose.

In some embodiments, the consortium comprises at least one anaerobic microbial cell population and at least one aerobic microbial cell population. For example, at least one aerobic microbial cell population may be Trichoderma reesei. In some embodiments, at least one anaerobic microbial cell population is a lactic acid bacterium. In some embodiments, at least one microbial cell population in the consortium is E. coli. In still other embodiments, each microbial cell population in the consortium is E. coli. In still other embodiments, at least one microbial cell population in the consortia is S. cerevisiae, Streptomyces spp., or Corynebacterium glutamicum.

In various embodiments, the chemical product is synthesized enzymatically by a recombinant biosynthetic pathway. In some embodiments, when using a consortium, the recombinant biosynthetic pathway is expressed across at least two or at least three or at least four microbial cell populations.

In some embodiments, the microbial strain expresses a heterologous biosynthetic pathway for producing the desired chemical product. Heterologous pathways for the production of terpene and terpenoid products are described in U.S. Pat. Nos. 11,028,413; 10,934,564; 10,501,780; 10,774,314; 10,463,062; US 2021/0254107, US 2021/0032669, and WO 2021/126960, each of which is hereby incorporated by reference in its entirety. Other heterologous pathways for producing desired chemical products are known in the art, examples of which are disclosed in U.S. Pat. Nos. 8,735,132 and 9,181,539; and U.S. pre-grant publications US 2022/0002764; US 2021/0261992; US 2021/0002678; and US 2019/0078098; each of which is hereby incorporated by reference in its entirety.

According to embodiments of this disclosure, the chemical product may be a terpene, terpenoid, alkaloid, cannabinoid, steroid, saponin, glycoside, stilbenoid, polyphenol, flavonoid, antibiotic, polyketide, fatty acid (e.g., short chain, medium chain, or long chain), triglyceride, peptide, or recombinant protein. In some embodiments, the chemical product is a terpene or terpenoid, such as a monoterpene or monoterpenoid, a sesquiterepene or sesquiterpenoid, or a diterpene or diterpenoid. In some embodiments, the product is an oil, e.g., comprising one or more terpenoids. In various embodiments, the disclosure allows for volatile organic products to be recovered without an organic overlayer. In still other embodiments, an organic overlayer is fed to the bioreactor system for continuous harvesting. An organic overlayer can be constructed as described in US 2021/0207078, which is hereby incorporated by reference in its entirety.

In various embodiments, the processes described herein can employ bacterial fermentation systems (e.g., including but not limited to E. coli). Such bacterial fermentation can produce products from the methylerythritol 4-phosphate (MEP) Pathway such as terpenes and terpenoids (e.g., monoterpenoids, sesquiterpentoids, diterpenoids, and triterpenoids). Bacterial strains engineered for higher MEP pathway flux (and fermentation methods employing the same) may be used, including those described in U.S. Pat. Nos. 8,512,988; 11,028,413; 10,662,442; 10,480,015; and 10,774,356, each of which is hereby incorporated by reference in its entirety. In some embodiments, the strain produces MEP pathway products (IPP and DMAPP) from prenol and/or isoprenol fed to the culture, as described in U.S. Pat. No. 11,034,980, which is hereby incorporated by reference. In some embodiments, the process employs yeast fermentation, and may include engineering of the mevalonic acid pathway (MVA) for higher flux to IPP and DMAPP and downstream metabolites such as terpenoids.

In various embodiments, the microbial strain is a bacterial strain, such as E. coli, that expresses one or more additional copies of one or more MEP pathway enzyme. The bacterial strain can be modified for improved carbon flux through the MEP pathway. For example, the strain can metabolize greater than 15% of the carbon entering glycolysis through the MEP pathway (greater than 15% “MEP carbon”), as described in U.S. Pat. No. 10,662,442, which is hereby incorporated by reference in its entirety. Such strains have increased availability or activity of Fe—S cluster proteins, so as to support higher activity of the Fe—S enzymes IspG and/or IspH. Modifications can include altering expression of the isc operon (by deleting iscR, with constitutive expression of the remaining genes of the operon), and/or by deletion of the ryhB small RNA. In addition, expression or activity of one or more competing enzymes or pathways, such as the ubiquinone synthesis pathway, can be tuned down. Further, by complementing IspG and/or IspH enzymes, strains can exhibit increased MEP carbon by pulling carbon further down the MEP pathway. Further still, MEP enzyme complementation can be balanced to provide for high MEP carbon, with carbon pulled further down the pathway to the MEcPP intermediate (the substrate for IspG).

In some embodiments, the bacterial strains overexpress IspG and IspH, so as to provide increased carbon flux to 1-hydroxy-2-methyl-2-(E)-butenyl 4-diphosphate (HMBPP) intermediate, but with balanced expression to prevent accumulation of HMBPP at an amount that reduces cell growth or viability, or at an amount that inhibits MEP pathway flux and/or terpenoid production. See, U.S. Pat. No. 10,480,015, which is hereby incorporated by reference in its entirety. The bacterial strain may include one or more genetic modifications to support the activities of IspG and IspH enzymes, which are Fe-sulfur cluster enzymes. Exemplary modifications include those that enhance the supply and transfer of electrons through the MEP pathway, and/or to terpene or terpenoid products. These include recombinant expression of one or more oxidoreductase enzymes, including oxidoreductases that oxidize pyruvate and/or lead to reduction of ferredoxin (which supplies electrons to the MEP pathway). An exemplary oxidoreductase is E. coli YdbK and its orthologs. In various embodiments, the microbial strain further comprises an overexpression of or complementation with one or more of a flavodoxin (fldA), flavodoxin reductase, ferredoxin (fdx), and ferredoxin reductase.

Synthesis of terpenes and terpenoids proceeds via conversion of IPP and DMAPP precursors to geranyl diphosphate (GPP), farnesyl diphosphate (FPP), or geranylgeranyl diphosphate (GGPP), farnesyl geranyl diphosphate (FGPP), through the action of a prenyl transferase enzyme (e.g., GPPS, FPPS, GGPPS, or FGPPS). Such enzymes are known, and are described for example in U.S. Pat. No. 8,927,241, WO 2016/073740, and WO 2016/029153, which are hereby incorporated by reference in their entireties. In some embodiments, these enzymes are heterologously expressed.

In various embodiments, the microbial strain expresses one or more terpenoid synthase enzymes, many of which are known in the art. Examples include those described in U.S. Pat. Nos. 10,227,597; 10,463,062; 10,934,564; 11,618,908, and published application nos. US 2021/0032669 and WO 2022/046994, which are hereby incorporated by reference in their entireties.

In various embodiments, the microbial strain expresses one or more P450 enzymes synthesizing the chemical product, and optionally one or more reductase partners. Numerous P450 oxygenase enzymes are known in the art, and include those described in U.S. Pat. Nos. 10,227,597; 10,463,062; 10,934,564; 10,774,314; 11,618,908 and published application nos. US 2021/0032669, US 2023/0042171, and WO 2022/046994, which are hereby incorporated by reference in their entireties. In some embodiments, for bacterial expression of a P450 hydroxylase and/or a reductase partner, the enzymes can be engineered for functional expression in bacterial cells (e.g., E. coli), as described in U.S. Pat. No. 10,774,314, which is hereby incorporated by reference in its entirety. For example, the engineered P450 enzymes can have a deletion of all or part of the wild type P450 N-terminal transmembrane region, and the addition of a transmembrane domain derived from a bacterial inner membrane, cytoplasmic C-terminus protein. It is believed that the transmembrane domain acts to anchor the P450 in the bacterial (e.g., E. coli) inner membrane. In some embodiments, the transmembrane domain (or “N-terminal anchor”) is derived from an E. coli gene selected from waaA, ypfN, yhcB, yhbM, yhhm, zipA, ycgG, djIA, sohB, 1pxK, F11O, motA, htpx, pgaC, ygdD, hemr, and ycls, or ortholog thereof.

In various embodiments, the product is an oil. For example, the product may comprise one or more components of an essential oil selected from ylang-ylang, tuberose, rose, neroli, mimosa, jasmine, champaca, bergamot, grapefruit, lemon, lime, orange, sandal wood, petitgrain, patchouli, pepper, costus, cypress, agarwood, jatamansi, kapoor kachri, saffron, ambrette, clary sage, orris, vanilla, and vetiver.

For example, the product may comprise a terpene or terpenoid selected from (−)-khusimone, (−)-limonene, (−)-methyl-(1R, 2R, 5S)-khusimal, (−)-methyl-(1R, 2S, 5S)-khusimal, (−)-rotundone, (+)-aromadendrene, (+)-khusimone, (+)-limonene, (+)-nootkatone, (1R, 2R, 5S)-khusimal, (1R, 2S, 5S)-khusimal, 1,4-cineole, 10-epi-gamma-eudesmol, 4-carvomenthenol, 4-terpineol, abietadiene, abietic acid, acetyl beta-caryophyllene, agarofuran, agarospirol, alpha pinene, alpha-bisabolol, alpha-cedrene, alpha-copaene, alpha-copaene-11-ol, alpha-damascone, alpha-eudesmol, alpha-funebrene, alpha-guaiene, alpha-gurjunene, alpha-humulene, alpha-santalene, alpha-santalol, alpha-selinene, alpha-sinensal, alpha-terpineol, alpha-terpinolene, alpha-vetivone, ambroxan/ambrein, amorphadiene, aristolene, aromadendrene, artemisinic acid, asiatic acid, astaxanthin, atisane, bergamotene, beta pinene, beta-bisabolene, beta-bisabolol, beta-carotene, beta-caryophyllene, beta-damascone, beta-eudesmol, beta-guaiene, beta-santalene, beta-santalol, beta-sinensal, beta-thujone, beta-vetivenene, beta-vetivone, beta-ylangene, bisabolol, boswelic acid, camphene, camphor, carvacrol, carveol, carvone, caryophyllene oxide, cedrenes, celastrol, cembrene, ceroplastol, cineol, cis-abienol, citral, citronellal, citronellol, copalol, cubebol, cucurbitane, cyperene, cyperene epoxide, cyperotundone, damascenone, dehydrofukinone, delta-cadinene, delta-damascone, delta-guaiene, delta-selinene, dihydro agarofuran, E-alpha-bisabolene, E-gamma-bisabolene, eleutherobin, epi-b-santalol, epi-zizaene, epi-zizaenone, eugenol, evopimaradene, farnescene, farnesol, fenchone, forskolin, gamma-bisabolol, gamma-cadinene, gamma-eudesmol, gamma-gurjunene, gamma-humulene, gamma-muurolene, gamma-terpinene, gascardic acid, geraniol, geranylgeraniol, germacrene D, glycyrrhizin, guaiol, guaiene, haslene, ionones, iripallidal, irones, isoborneol, isopemaradiene, isoprene, iso-velencenol, jinkohols, karanone, kaurene, kessane, khusimene, khusimol, labdenediol, ledene, ledol, levopimaradiene, levopimaric acid, linalool, linalool oxide, longifolenaldehyde, longipinene, L-rose oxide, lupeol, madeccasic acid, menthol, menthone, methyl vetivenate, mogrosides, muurolenes, myrcene, nerolidol, nootkatene, nootkatol, nootkatone, ocimenes, ophiobolin A, patchouli alcohol, pinene, piperitone, pogostol, prenol, protopanaxadiol, protopanaxatriol, pulegone, R-(−)-carvone, rotundone, S-(+)-carvone, sabinene, sabinene hydrate, santalals, santalenes, santalols, sclarene, sclareol, selina-3, 7(11)-diene, selinadiene, spathulenol, steviol, steviol glycosides, sulcatone, tagetone, taxadiene, thymol, ursolic acid, valencene, valeranone, verbenone, vetiverol, vetiverone, vetiveryl acetate, viridiflorol, Z, E-alpha-bergamotol, Z-alpha-bisabolene, zeaxanthin, Z-gamma-bisabolene, zizaene, zizenone, and Z-lanceol.

In various embodiments, a microbial cell population expresses heterologous gene encoding one or more of a uridine diphosphate dependent glycosyltransferase enzyme (UGT), a methyltransferase enzyme, a acetyltransferase enzyme, or a benzoyl transferase enzyme. For example, in embodiments employing a UGT enzyme, the chemical product can be selected from a terpenoid glycoside, flavonoid glycoside, cannabinoid glycoside, polyketide glycoside, stilbenoid glycoside, and polyphenol glycoside.

In some embodiments, the desired chemical product is produced by whole cell bioconversion of a fed substrate. Such bioconversion processes include glycosylations of a fed substrate (including but not limited to terpenoid glycosides such as steviol glycosides and mogrosides) as described in U.S. Pat. Nos. 11,230,724 and 11,168,309, which are hereby incorporated by reference in their entireties. For example, the chemical product can be a terpenoid glycoside, such as a steviol glycoside or mogrol glycoside. In such embodiments, the fed substrate is a plant extract, and which is optionally a stevia leaf extract or fraction thereof, or a monkfruit extract or fraction thereof.

The biomass retention process described herein can employ bioreactors with mechanical agitation (e.g., a stirred tank reactor). Alternatively, the process can employ bioreactors that do not involve mechanical agitation, such as a bubble column reactor.

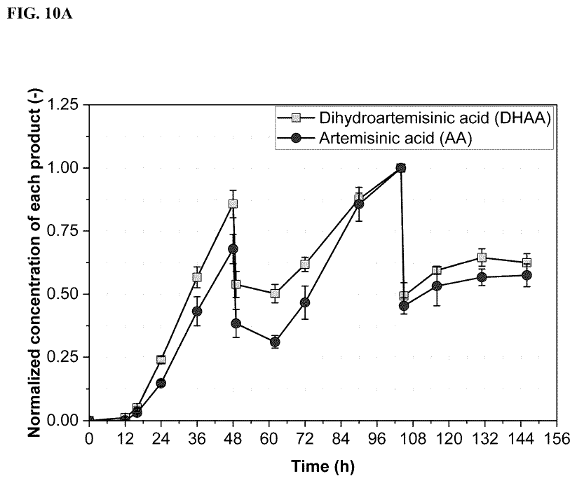

In other aspects, this disclosure provides a fermentation system for conducting the method disclosed herein. Exemplary fermentation systems are illustrated in FIGS. 11 to 15. In some embodiments employing in situ filtration, the invention provides a fermentation system comprising a bioreactor vessel containing growth media and a microbial biomass. The system further comprises: a filtration membrane removing permeate from the bioreactor vessel in situ while retaining biomass (including as described herein); a feeding vessel comprising fresh broth fluidly connected to the bioreactor vessel; and a vessel for collecting filtrated broth fluidly connected to the filtration membrane. In some embodiments involving ex situ filtration of fermentation broth, the invention provides a fermentation system comprising a bioreactor vessel containing growth media and a microbial biomass. The system further comprising: a cross filtration module (CFM) fluidly connected to the bioreactor vessel, the CFM comprising a membrane removing permeate from the growth media ex situ while retaining biomass; a feeding vessel comprising fresh broth fluidly connected to the bioreactor vessel; and a vessel fluidly connected to the CFM for collecting filtrated broth, while retentate comprising biomass is recirculated to the bioreactor via a recirculation line.

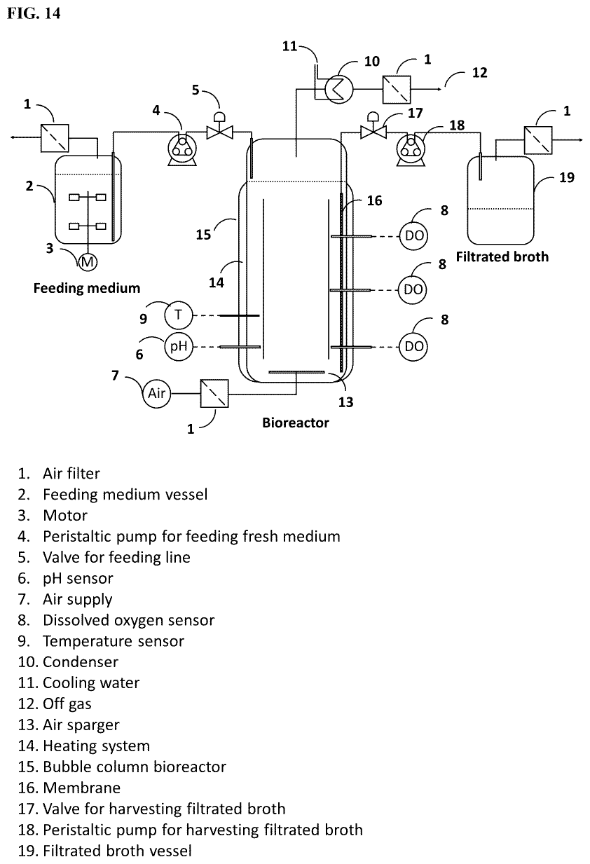

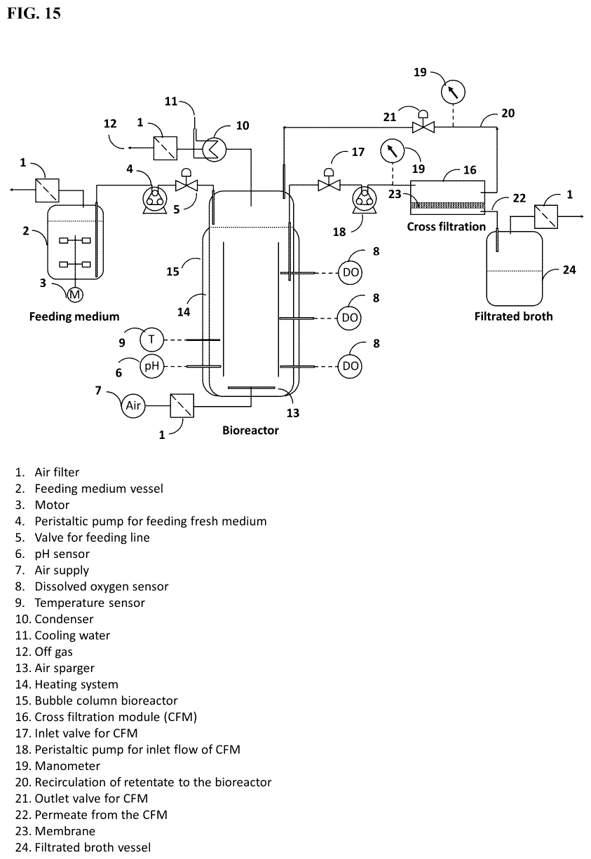

FIG. 11 illustrates a stirred tank reactor design (mechanical agitation) with in situ extraction of fermentation media. The bioreactor (stirred-tank) (15) is shown comprising a motor (3), air sparger (13), and heating system (14). The bioreactor further comprises a tubular filtration membrane (16) removing permeate while retaining biomass. The bioreactor is fed fresh broth using feeding vessel (2), also comprising a motor (3) for stirring. Fresh broth is fed in controlled manner using a peristaltic pump (4), and through feeding line valve (5). Media and cells are removed from the bioreactor by peristaltic pump (18) through valve (17), with the line operably connected to filtrated broth vessel (19). The bioreactor system further comprises pH sensor (6), air supply (7) (with air filter (1)), dissolved oxygen sensor (8), and temperature sensor (9). FIG. 14 illustrates a similar bioreactor system employing a bubble column reactor (non-mechanically agitated) with in situ extraction of fermentation media.

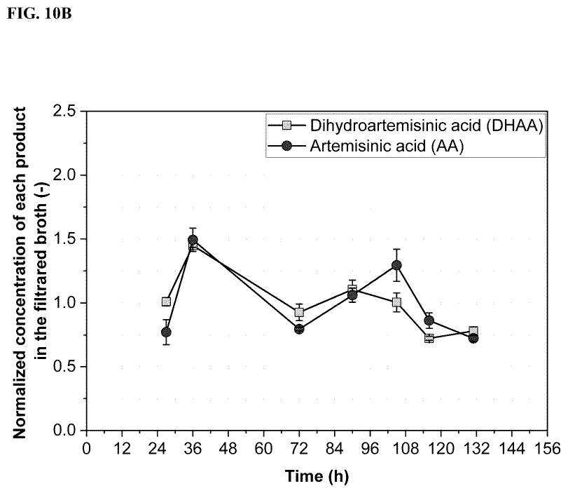

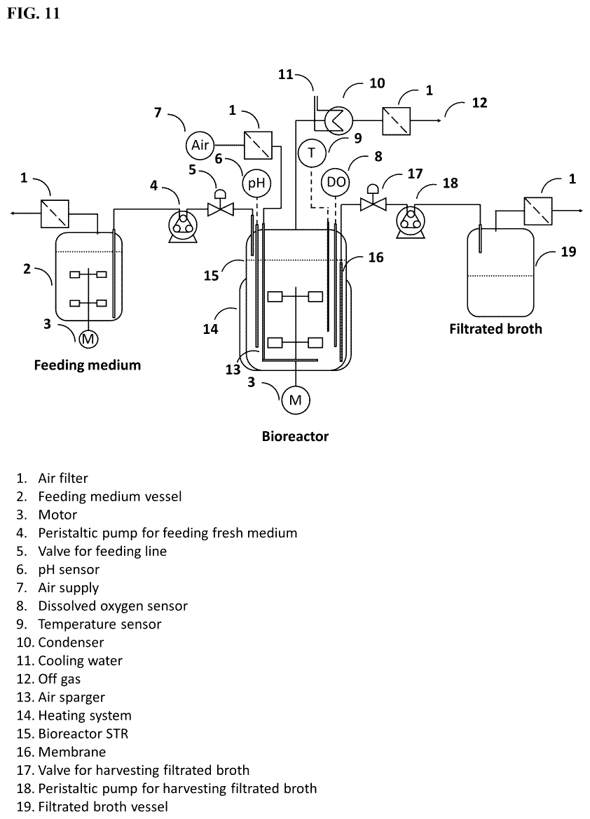

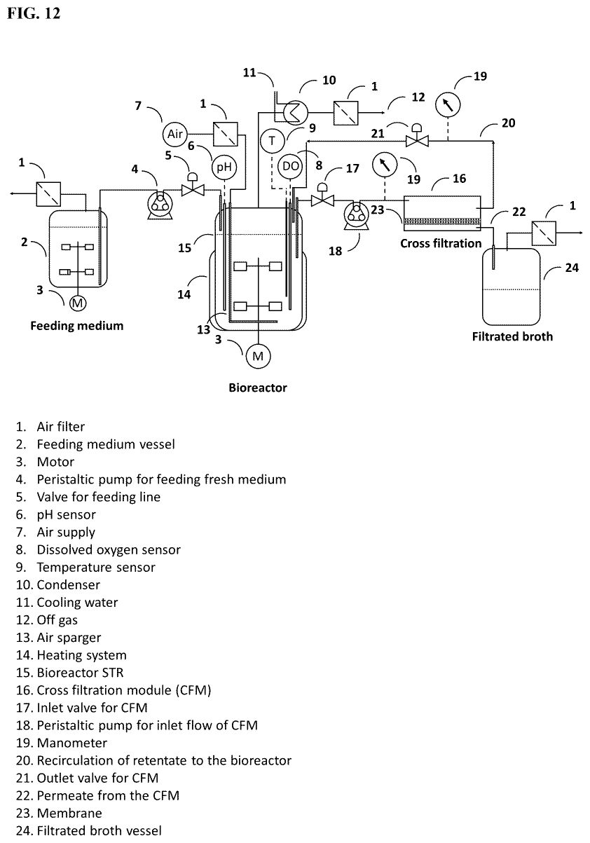

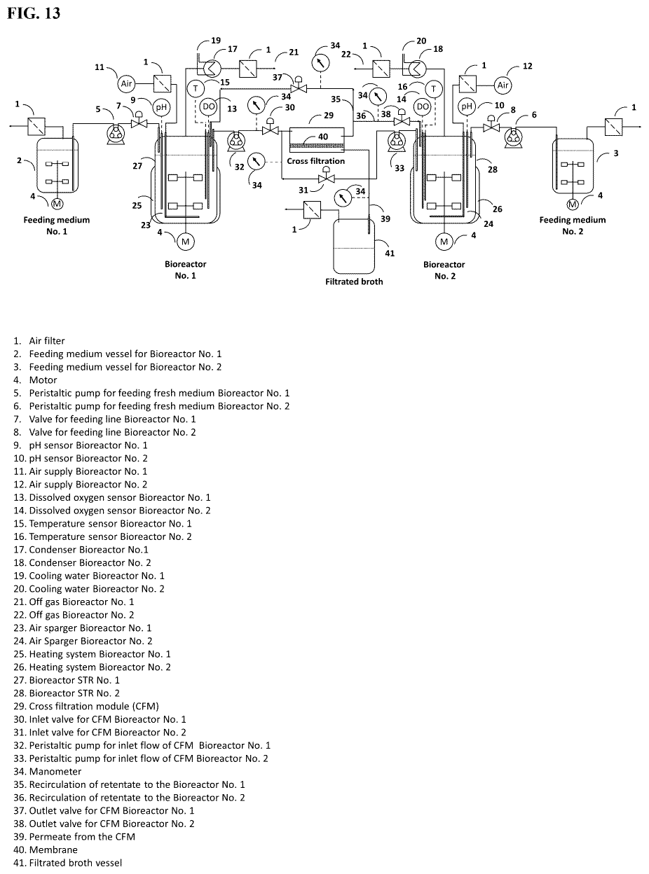

FIG. 12 illustrates a stirred tank reactor design (mechanical agitation) and an external (ex situ) extraction module. The bioreactor (stirred-tank) (15) is shown comprising a motor (3), air sparger (13), and heating system (14). The bioreactor is fed fresh broth using feeding vessel (2), also comprising a motor (3) for stirring. Fresh broth is fed in controlled manner using a peristaltic pump (4) and through feeding line valve (5). Media and cells are removed from the bioreactor by peristaltic pump (18) through valve (17), the line being operably connected to a cross filtration module (CFM) (16) with membrane (23). Permeate from the CFM (22) is sequestered in the filtrated broth vessel (24), while retentate (including biomass) is recirculated to the bioreactor. The bioreactor system further comprises pH sensor (6), air supply (7) (with air filter (1)), dissolved oxygen sensor (8), and temperature sensor (9). FIG. 13 illustrates a system, such as that of FIG. 12), but employing multiple stirred tank reactors (mechanical agitation) connected to an external (ex situ) extraction module. FIG. 15 illustrates bioreactor system such as shown in FIG. 12, but employing a bubble column reactor (non-mechanically agitated) with an external (ex situ) extraction module.

As used herein, the terms “about” or “approximately” mean±10% of an associated value, unless the context requires otherwise.

In still other aspects, the disclosure provides methods for microbial fermentation that limit and/or avoid the use of yeast extract and other complex media for creating biomass and/or for the production phase, since yeast extract is costly and can complicate downstream processing. In this aspect, the microbial strains as described herein (including but not limited to bacterial strains such as E. coli) are cultured in fed-batch, semi-continuous, or continuous systems with yeast extract partly or completely replaced with a defined media comprising an amino acid mix. In some embodiments, the defined media further comprises supplementation with pyruvate. For example, the production phase comprises feeding a nitrogen source and a carbon source. For example, the nitrogen source can comprise ammonium (e.g., ammonium hydroxide). The carbon source may contain carbon sources such as C1, C2, C3, C4, C5, and/or C6 carbon sources. Exemplary carbon sources are glucose, glycerol, and sucrose (and are defined herein). The nitrogen and carbon feeding can be initiated once sufficient biomass is created, or in some embodiments is also used for creation of biomass (e.g., in the absence of yeast extract or other base media). In various embodiments, the media for the production phase comprises at least amino acids that are synthesized from pyruvate, such as L-Ala, L-Val, and L-Leu. In some embodiments, the amino acid mix can replace yeast extract partially or completely. An exemplary amino acid mix (used herein for an E. coli culture) comprises, consists essentially of, or consists of L-Ala, L-Glu, Gly, L-Leu, L-Val, and L-Ile. Various amino acid mixes are known in the art and can be selected or tailored for the microbial species or strain. For example, 1, 2, 3, 4, or 5 additional amino acids can be added to support strain growth and/or metabolism. In some embodiments, amino acid mix is employed together with yeast extract. In some embodiments, amino acids are fed to the system for the creation of biomass or during the production phase without yeast extract. In embodiments that avoid substantial use of yeast extract, downstream processing can be facilitated, in view of the complex nature of yeast extract.

Further aspects and embodiments of the invention will be apparent from the following, non-limiting working examples.

### EXAMPLES

The following studies evaluate the application of a biomass retention process with single and multiple cycles in the production of the terpenoid amorpha-4,11-diene via the methylerythritol 4-phosphate pathway (MEP pathway) in E. coli. For a single cycle of a biomass retention process, 50% of the fermented broth is continually removed and replaced with fresh medium (e.g., over about 42 h period). The biomass retention employed a stirred tank reactor with a coupled tubular filtration probe (polypropylene, pore size 0.2 microns) for in situ extraction of fermented broth. External peristaltic pumps with sterilized feeding lines were used for media exchange, pH and nitrogen concentration control, and media feeding. Flow rates were adjusted based on multiple weighing scale readings. Process parameters such as dissolved oxygen, agitation speed, temperature, and aeration rate were controlled by a PID controller. Exhaust gas was sterile filtered and transferred into an off-gas analyzer for in-line monitoring.

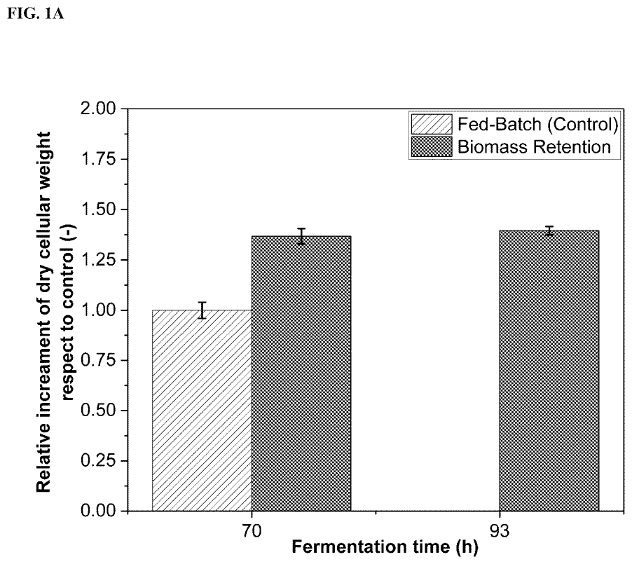

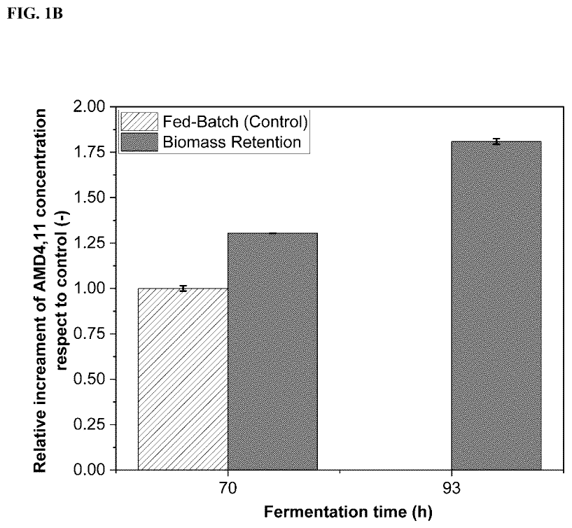

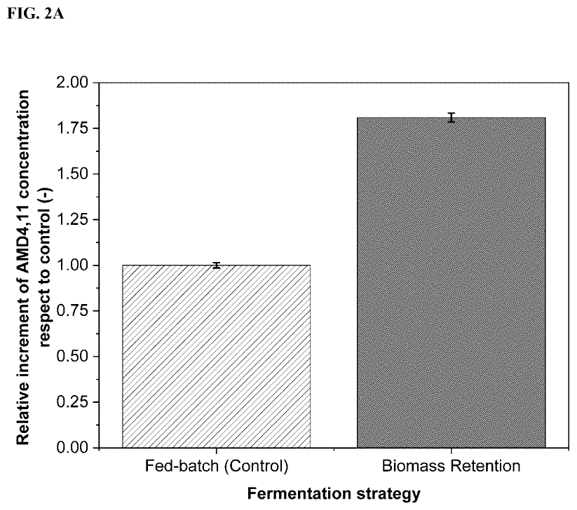

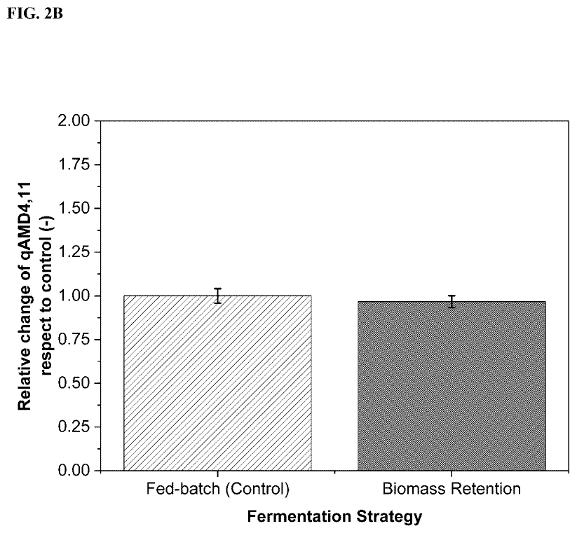

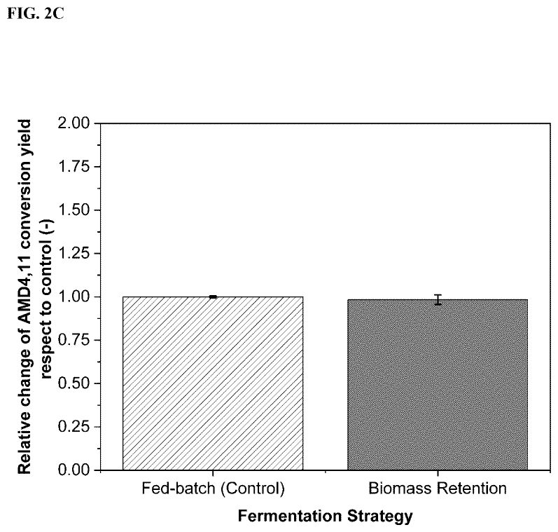

With a single biomass retention cycle under carbon limiting conditions, it was possible to increase the dry cellular weight by about 40% without reducing the AMD4,11-specific biomass production rate or conversion yield, which increased the product concentration up to about 80% and decreased the process time by about 26%, in comparison to a traditional fed-batch process using as a calculation basis the total amount of AMD4,11. See FIGS. 1A, B and FIGS. 2A-C.

FIG. 1B shows relative AMD4,11 concentration during the fermentation course. The biomass retention process resulted in over 80% improvement in product with extended production times. FIG. 2A-C compare biomass specific productivity using the biomass retention process and a fed-batch process. FIG. 2A shows the relative final amorpha-4,11-diene production. FIG. 2B shows the relative AMD4,11 production rate between fed-batch and the biomass retention strategy, and FIG. 2C shows AMD4,11 conversion yield between fed-batch and the biomass retention strategy.

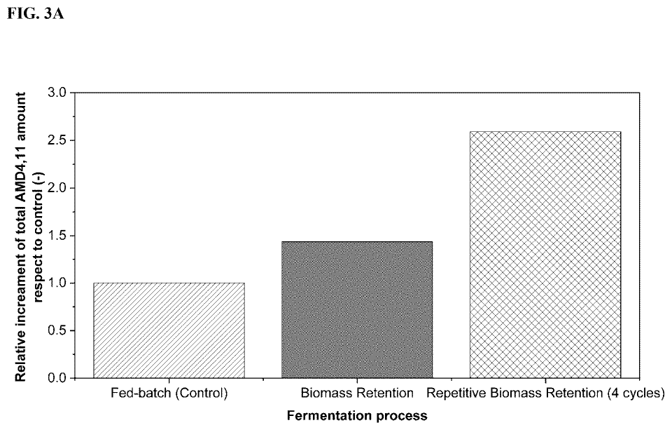

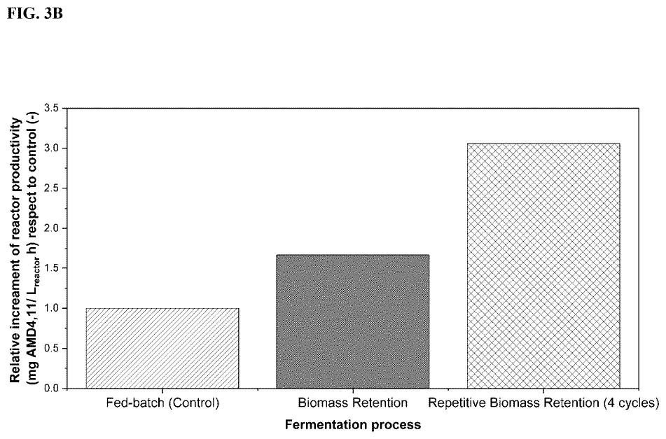

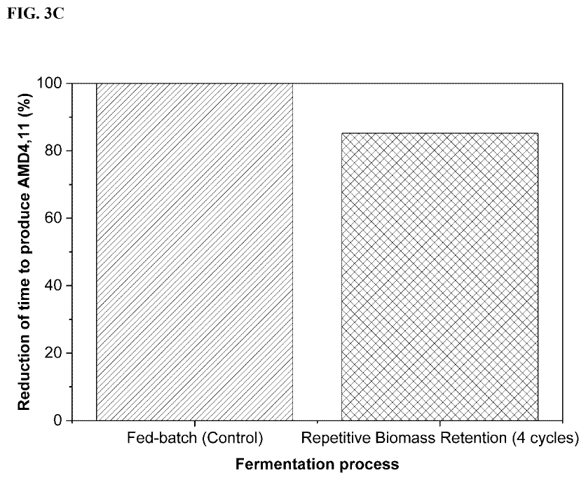

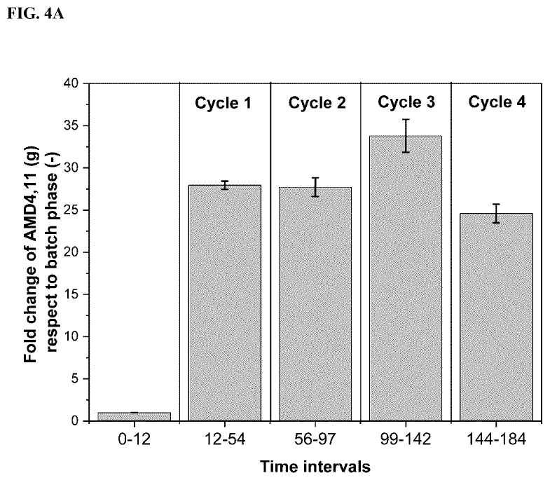

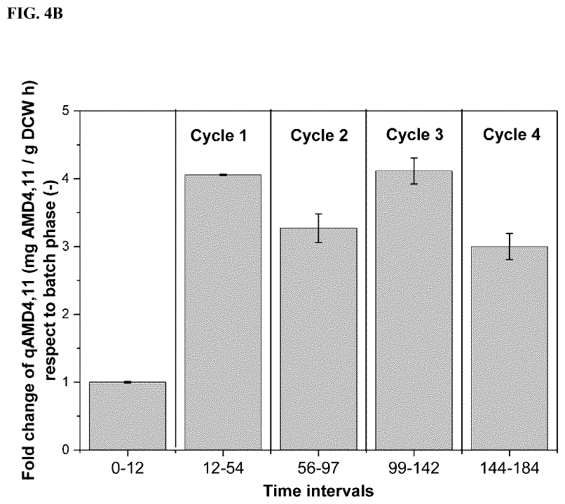

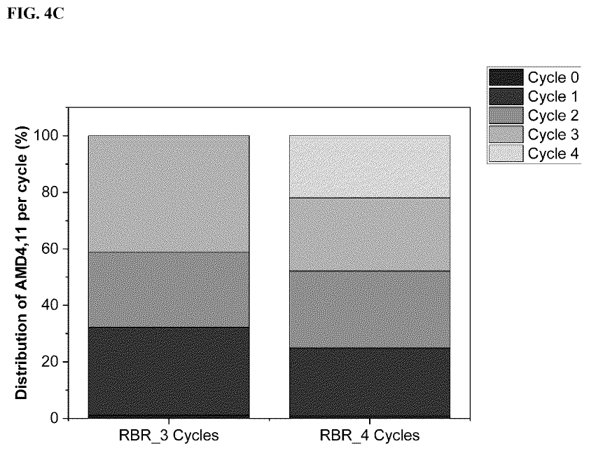

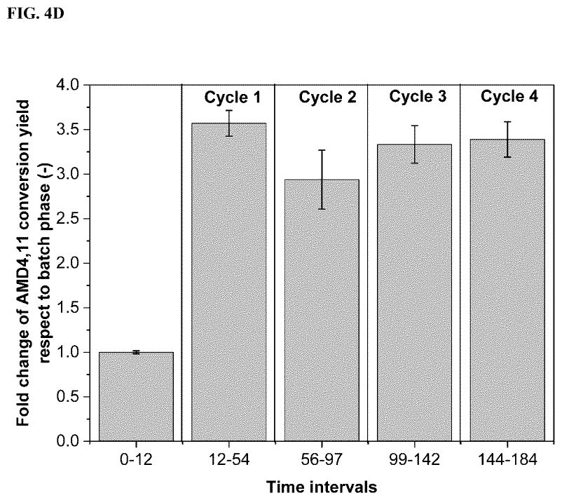

To increase the reactor productivity of the process, a repetitive biomass retention mode was executed, replacing 50% of the fermented broth with fresh medium approximately each 42 h. As a result, the variation between the batch productivity (mg/h) of each cycle was lower than 10%, limiting impacts on the production capacity of the strain by a medium exchange. After four cycles, the total amount of AMD4,11 was enhanced by 1.43-fold and 2.59-fold concerning single biomass retention and fed-batch process. Reactor productivity showed the same trend with a fold change of 1.66 and 2.95, respectively. The bioreactor productivity was not significantly diminished after four cycles. In addition, the time to produce the amount of AMD4,11 reached by repetitive biomass retention using a regular fed-batch was also reduced by 14.81%. See FIGS. 3A-C and FIGS. 4A-4D.

FIG. 3A-C show the incremental improvements in AMD4,11 production using a single cycle or repetitive biomass retention process, as compared to a fed batch process. FIG. 3A shows total AMD4,11 produced. FIG. 3B shows reactor productivity. FIG. 3C shows reductions in time using the repetitive biomass retention approach, as compared to fed-batch. FIG. 4A-D illustrates AMD4,11 productivity using a biomass retention approach. FIGS. 4A and 4B show fold change in AMD4,11 production across 4 cycles of media exchange in a repetitive biomass retention process. FIG. 4C shows distribution of AMD4,11 produced per cycle in a 3 and 4 cycle process. FIG. 4D shows AMD4,11 conversion yield across 4 cycles of media exchange in a repetitive biomass retention process.

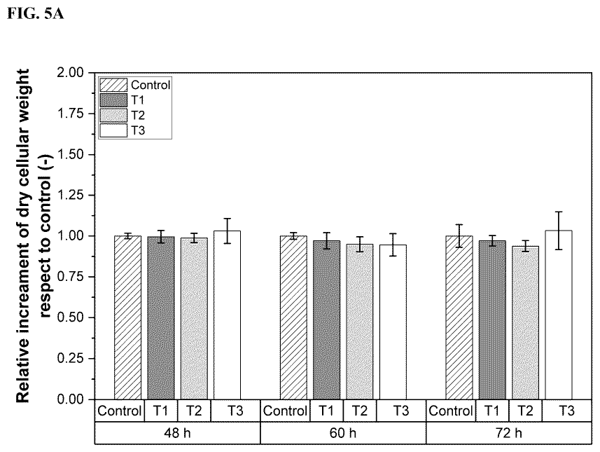

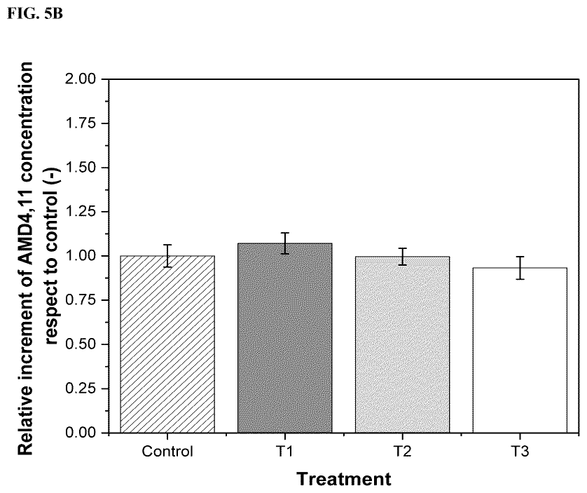

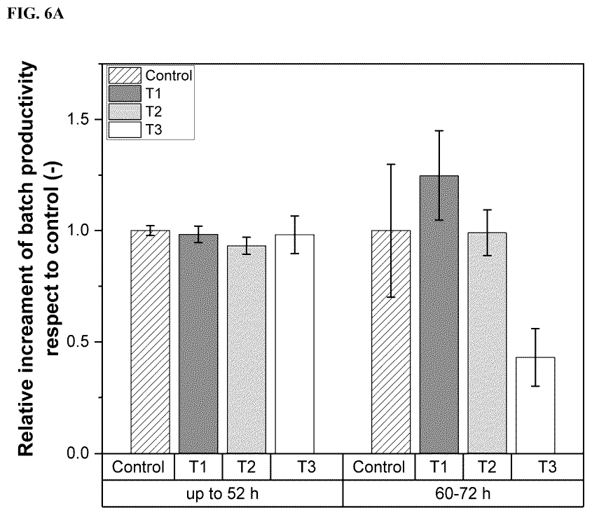

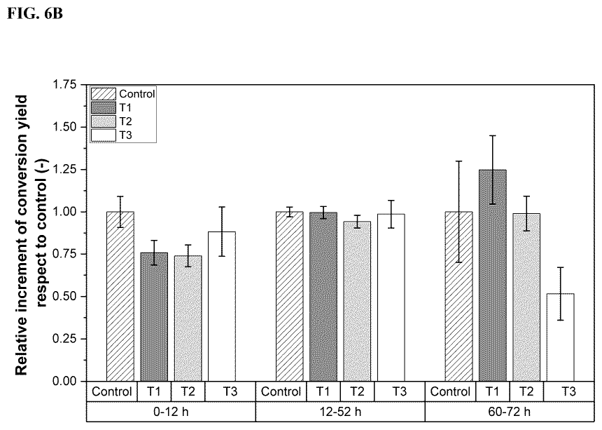

Studies were undertaken with chemically defined media to evaluate the effect of pyruvate supplementation during the stationary phase (i.e., the production phase). These studies evaluated media supplementation using a fed-batch process. It was determined that productivity increased by about 30% with only 2% pyruvate supplementation of glucose (based on total carbon). An optimum concentration of pyruvate was about 0.09 g/h. See FIGS. 5A,B and FIGS. 6A,B.

FIG. 5A shows dry cellular weight (g/L) during a fed-batch fermentation with pyruvate supplementation (T1=0.09 g/h; T2=0.18 g/h; T3=0.27 g/h) with respect to a control treatment in different sampling point (48, 60, 72 h). FIG. 5B shows total amount of AMD4,11 produced during a fed-batch fermentation with pyruvate supplementation (T1=0.09 g/h; T2=0.18 g/h; T3=0.27 g/h) with respect to a control treatment. FIG. 6A shows batch productivity (mg/h) during a fed-batch fermentation with pyruvate supplementation (T1=g/h; T2=0.18 g/h; and T3=0.27 g/h) with respect to a control treatment in two time frames, up to 52 h and 60-72 h. FIG. 6B shows conversion yield (mg AMD4,11/g glucose) during a fed-batch fermentation with pyruvate supplementation (T1=0.09 g/h; T2=0.18 g/h; T3=0.27 g/h) with respect to a control treatment in three time frames: 0-12 h (batch phase), 12-52 h (fed-batch without supplementation), and 60-72 h (fed-batch with supplementation of pyruvate). Particular improvements are seen in the 60-72 h window.

Studies were undertaken to compare the use of yeast extract with amino acid mix, using the repetitive biomass retention process. The following media compositions were tested:

TABLE 1
Media Compositions

| | Base | Yeast | AA |
|---|---|---|---|
| | medium | extract (g/L) | mix (Yes/No) |
| RBR-control | FM06 | 20 | No |
| RBR-10YE + AA | FM06 | 10 | Yes |
| RBR-5YE + AA | FM06 | 5 | Yes |
| RBR + AA | FM06 | 0 | Yes |

The amino acid mix was defined as follows: L-Ala (900.5 mg/L), L-Glu (1436.5 mg/L), Gly (262 mg/L), L-Leu (910 mg/L), L-Val (515 mg/L), and L-Ile (546.5 mg/L). The base media had the following composition: (NH₄)₂SO₄ (4.4 g/L), KH₂PO₄ (14.6 g/L), K₂HPO₄ (5.2 g/L), citric acid (1.2 g/L), yeast extract (Table 1), FeSO₄7H₂O (0.352 mM), MgSO₄ (3.52 mM), Thiamine HCl (3.52 mM), trace elements.

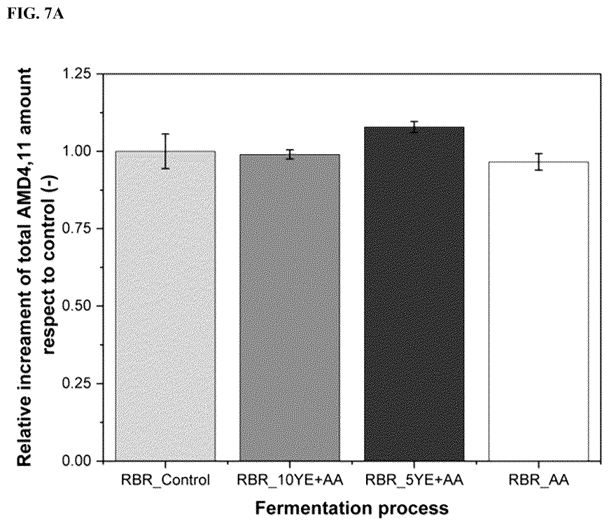

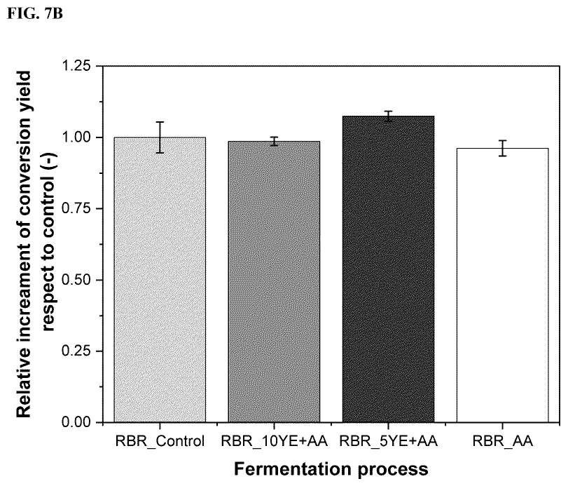

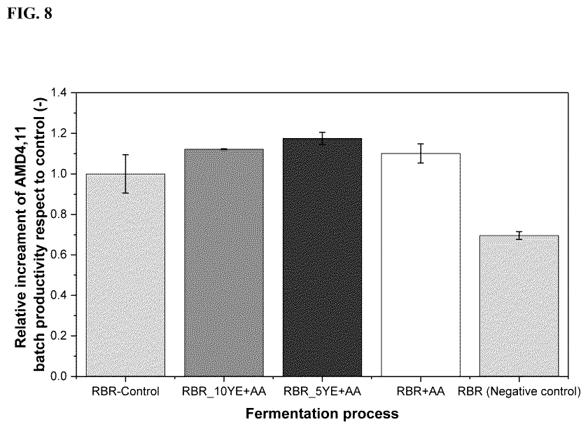

Results are shown in FIGS. 7A,B and FIG. 8. FIG. 7A shows the total amount of AMD4,11 produced during a repetitive biomass retention (RBR) process using the media compositions (n=2). FIG. 7B shows conversion yield to AMD4,11. FIG. 8 shows AMD4,11 batch productivity during a repetitive biomass retention process using different media composition (n=2). As shown, the amino acid mix is able to replace yeast extract.

These results demonstrate a biomass retention process producing the sesquiterpene amorpha-4,11-diene. These processes are applicable to other products, including other terpenoids and other secondary metabolites (including natural compounds) and recombinant protein. The processes described herein are applicable to various microbial fermentation or whole cell bioconversion system, including bacterial and yeast systems. In various embodiments, the invention can reduce process time associated with preparation activities (cleaning and sterilization) or inoculum production and increase reactor productivity due to the biomass concentration under optimal production conditions.

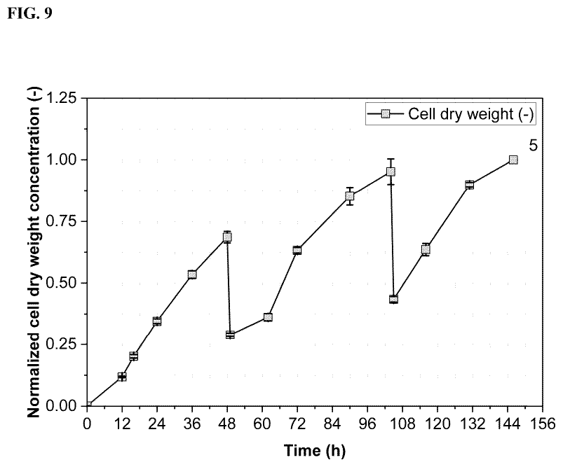

The following examples demonstrate the production of dihydroartemisinic acid (DHAA) and artemisinic acid (AA) using the RBR process. FIG. 9 shows cell dry weight concentration (normalized by the maximum achieved value) in a biomass retention process with multiple cycles with an AA-producing strain (DMD6). FIG. 10A shows DHAA and AA concentration normalized by the maximum achieved value in a biomass retention process by multiple cycles with the strain DMD6. The maximum DHAA and AA concentration was similar to the fed-batch process. FIG. 10B shows DHAA and AA concentration in the filtered broth normalized by the mean value (n=3). Both DHAA and AA were detected in the filtered broth from the filtration module, allowing for the possibility of a continuous production process.

The following references are hereby incorporated by reference in their entireties:
Paddon et al., High-level semi-synthetic production of the potent antimalarial artemisinin. Nature Vol. 496, 529 (2013).
Shukal S., et al., Systematic engineering for high-yield production of viridiflorol and amorphadiene in auxotrophic Escherichia coli. Metabolic Engineering 55:170-178 (2019).
Tsuruta H. et al., High-Level Production of Amorpha-4.11-Diene, a Precursor of the Antimalarial Agent Artemisinin, in Escherichia coli. PLOS One Vol. 4, Issue 2 e4489 (2009).
Westfall P., et al. Production of amorphadiene in yeast, and its conversion to dihydroartemisinic acid, precursor to the antimalarial agent artemisinin. PNAS Vol. 109, No. 3 E111-E118 (2012).

1. A method for making a chemical product by microbial fermentation, comprising:
providing a cell culture producing the chemical product from carbon substrate or by enzymatic bioconversion of a fed substrate, each optionally through one or more heterologously expressed enzymes;
culturing the cell in a fermentation media in a bioreactor under conditions suitable for producing the chemical product, while continuously harvesting filtered fermentation media from the bioreactor and feeding the culture with fresh media while retaining cultured biomass; and
recovering the chemical product from the filtered fermentation media and/or the retentate.

2. The method of claim 1, wherein the chemical product is recovered from the filtered media.

3. The method of claim 1, wherein the chemical product is recovered from the retentate.

4. The method of claim 1, wherein the chemical product is recovered from both the filtered media and the retentate.

5. The method of any one of claims 1 to 4, wherein the volume of the fermentation media in the bioreactor is kept substantially constant, and in some embodiments does not vary by more than about 15%, or more than about 10%, or more than about 5%, during a production phase.

6. The method of claim 5, wherein the production rate of the chemical product is kept substantially constant, and in some embodiments does not vary by more than about 20%, or more than about 15%, or more than about 10% during the production phase.

7. The method of claim 6, wherein the productive phase is maintained for at least about 72 hours, or at least about 96 hours, or at least about 120 hours, or at least about 144 hours, or at least about 168 hours, or at least about 192 hours, or at least about 216 hours, or at least about 240 hours, or at least about 288 hours, or at least about 336 hours.

8. The method of claim 6 or claim 7, wherein the production phase involves carbon limiting conditions, and wherein the media comprises a nitrogen source and a carbon source.

9. The method of claim 8, wherein the carbon source comprises one or more C1 to C6 carbon sources.

10. The method of claim 8, wherein the carbon source comprises one or more of glucose, sucrose, fructose, xylose, and glycerol.

11. The method of claim 8, wherein the fresh media comprises yeast extract and/or amino acids.

12. The method of any one of claims 1 to 11, wherein the fresh media is supplemented with pyruvate.

13. The method of claim 8, wherein the carbon source comprises an organic stream selected from lignocellulosic biomass or municipal waste, or a carbon source derived therefrom.

14. The method of claim 8, wherein the carbon source comprises CO₂ or biogas generated by anaerobic digestion, and which optionally comprises methane.

15. The method of any one of claims 5 to 14, wherein the levels of media components and pH are kept substantially constant during the production phase.

16. The method of claim 15, wherein concentration of nitrogen is kept substantially constant during the production phase.

17. The method of any one of claims 1 to 16, wherein the culture is maintained at high cell density, and in various embodiments, at least about 0.1 gram of dry cells per L, 1 gram of dry cells per L, or at least about 5 grams of dry cells per L, or at least about 8 grams of dry cells per L, or at least about 10 grams of dry cells per L, or at least about 12 grams of dry cells per L, or at least about 15 grams of dry cells per L, or at least about 20 grams of dry cells per L, or at least about 25 grams of dry cells per L, or at least about 30 grams of dry cells per L, or at least about 35 grams of dry cells per L, or at least about 40 grams of dry cells per L.

18. The method of any one of claims 1 to 17, wherein the volume of media in the bioreactor is replaced at least twice during the production phase, or at least four times during the production phase, or at least six times during the production phase, or at least eight times during the production phase, or at least ten times during the production phase, or at least 20 times during the production phase.

19. The method of claim 18, wherein every 24 hours at least about 5% of the media volume is replaced, or at least about 10% of the media volume is replaced, or at least about 15% of the media volume is replaced, or at least about 20% of the media volume is replaced, or at least about 25% of the media volume is replaced, or at least about 30% of the media volume is replaced, or at least about 35% of the media volume is replaced, or at least about 40% of the media volume is replaced, or at least about 45% of the media volume is replaced, or at least about 50% of the media volume is replaced.

20. The method of claim 19, wherein every 24 hours from 5% to about 50% of the media volume is replaced, or from about 10% to about 40% of the media volume is replaced, or from about 10% to about 30% of the media volume is replaced, or from about 15% to about 35% of the media volume is replaced, or about 25% of the media volume is replaced.

21. The method of any one of claims 1 to 20, wherein media is not replaced during an initial growth phase.

22. The method of any one of claims 18 to 21, wherein the volume of the media in the bioreactor is about 250,000 L or less, or about 150,000 L or less, or about 100,000 L or less, or is about 50,000 L or less, or is about 25,000 L or less, or is about 10,000 L or less, or is about 1000 L or less.

23. The method of claim 22, wherein the volume of the media in the bioreactor is from about 10,000 L to about 250,000 L, or from about 10,000 L to about 100,000 L.

24. The method of any one of claims 1 to 23, wherein the bioreactor comprises a filtration probe or an external filtration module filtering fermentation media from biomass.

25. The method of claim 24, wherein the filtration probe or filtration module comprises a hydrophobic or hydrophilic membrane filtering the fermentation media from biomass.

26. The method of claim 25, wherein the filtration probe or module comprises a polypropylene membrane filtering the fermentation media from biomass.

27. The method of claim 26, wherein the filtration probe or module comprises a polyethersulfone (PES) filter, a nylon filter, cellulose acetate filter, or cellulose nitrate filter.

28. The method of claim 25, wherein the filtration probe or module comprises a polyfluoroalkyl filter.

29. The method of any one of claims 1 to 28, wherein the filter is approximately a 0.2 micron filter.

30. The method of any one of claims 24 to 29, wherein the filtration probe or module is operated by a pump.

31. The method of any one of claims 1 to 30, wherein the cell is a bacterium.

32. The method of claim 31, wherein the bacterium belongs to a genus selected from Acidovorax, Acinetobacter, Actinomyces, Alcanivorax, Arthrobacter, Brevibacterium, Bacillus, Clostridium, Corynebacterium, Deinococcus, Dietzia, Escherichia, Gordonia, Marinobacter, Mycobacterium, Micrococcus, Micromonospora, Moraxella, Nocardia, Pseudomonas, Psychrobacter, Rhodobacter, Rhodococcus, Salmonella, Streptomyces, Thalassolituus, Thermomonospora, Vibrio, and Zymomonas.

33. The method of claim 32, wherein the bacterium is a species selected from Rhodococcus opacus, Acinetobacter calcoaceticus, Streptomyces coelicolor, Rhodococcus jostii, and Acinetobacter baylyi.

34. The method of claim 32, wherein the bacterium is a species of Escherichia, Bacillus, Corynebacterium, Deinococcus, Rhodobacter, Zymomonas, Pseudomonas, Vibrio, and Zymomonas.

35. The method of claim 34, wherein the bacterium is Escherichia coli, Bacillus subtilis, Corynebacterium glutamicum, Rhodobacter capsulatus, Rhodobacter sphaeroides, Zymomonas mobilis, Pseudomonas putida, and Vibrio natriegens.

36. The method of claim 35, wherein the bacterium is E. coli.

37. The method of any one of claims 1 to 30, wherein the microorganism is yeast.

38. The method of claim 37, wherein the yeast is a species of Ashbya, Aspergillus, Aurantiochytrium, Bastobotyrs, Candida, Claviceps, Cryptococcus, Cunninghamella, Geotrichum, Hansenula, Issatchenkia, Kluyveromyces, Kodamaea, Leucosporidiella, Linderna, Lipomyces, Mortierella, Myxozyma, Mucor, Occultifur, Ogataea, Penicillium, Phaffia, Pichia, Prototheca, Rhizopus, Rhodosporidium, Rhodotorula, Saccharomyces, Scheffersomyces, Schizosaccharomyces, Sporidiobolus, Sporobolomyces, Starmerella, Tremella, Trichosporon, Wickerhamomyces, Waltomyces, and Yarrowia.

39. The method of claim 38, wherein the yeast or fungal cell is a species selected from Yarrowia lipolytica, Yarrowia phangngensis, Pichia kudriavzevii, Saccharomyces cerevisiae, Pichia pastoris, Kluyveromyces marxianus, Rhodosporidium toruloides, Sporidiobolus ruinenii, Sporidiobolus salmonicolor, Aspergillus oryzae, Mortierella isabellina, Waltomyces lipofer, Candida tropicalis, Candida boidinii, Scheffersomyces stipitis, Mucor circinelloides, Ashbya gossypii, Trichoderma harzianum, Pichia guilliermondii, Kodamaea ohmeri, Rhodotorula aurantiaca, Lindnera saturnus, Penicillium roqueforti, Lipomyces starkeyi, and Bastobotyrs adeninivorans.

40. The method of claim 37, wherein the yeast cell is selected from a species of Saccharomyces, Pichia, or Yarrowia.

41. The method of claim 37, wherein the yeast cell is Saccharomyces cerevisiae, Pichia pastoris, and Yarrowia lipolytica.

42. The method of any one of claims 1 to 30, wherein the cell uses CO₂ as a carbon source, and is optionally an algae, cyanobacteria that is optionally Synechoccus elongatus, or acetogenic bacteria.

43. The method of claim 42, wherein the algae is microalgae.

44. The method of claim 42 or 43, wherein the bioreactor design allows the microbial cells to be exposed to sunlight.

45. The method of any one of claims 1 to 30, wherein the culture comprises a consortium of microbial cells.

46. The method of claim 45, wherein the consortium comprises at least a first microbial cell population that converts a carbon source to an intermediary metabolite, and one or more additional microbial cell populations that convert the intermediary metabolite to the chemical product.

47. The method of claim 46, wherein the first microbial cell population is a cyanobacteria that is optionally Synechoccus elongatus, and which produces sucrose from a CO₂ carbon source; and wherein the one or more additional microbial cell populations produce the chemical product from sucrose.

48. The method of claim 47, wherein the consortium comprises at least one anaerobic microbial cell population and at least one aerobic microbial cell population.

49. The method of claim 48, wherein at least one aerobic microbial cell population is Trichoderma reesei.

50. The method of claim 48 or 49, wherein at least one anaerobic microbial cell population is a lactic acid bacterium.

51. The method of claim 45 or 46, wherein at least one microbial cell population in the consortium is E. coli.

52. The method of claim 51, wherein each microbial cell population in the consortium is E. coli.

53. The method of claim 51, wherein at least one microbial cell population in the consortia is S. cerevisiae, Streptomyces spp., or Corynebacterium glutamicum.

54. The method of any one of claims 45 to 53, wherein the chemical product is synthesized enzymatically by a recombinant biosynthetic pathway, the recombinant biosynthetic pathway expressed across at least two or at least three or at least four microbial cell populations in the consortia.

55. The method of any one of claims 1 to 54, wherein the chemical product is a terpene, terpenoid, alkaloid, cannabinoid, steroid, saponin, glycoside, stilbenoid, polyphenol, flavonoid, antibiotic, polyketide, fatty acid, peptide, or recombinant protein.

56. The method of claim 55, wherein the chemical product is a terpene or terpenoid.

57. The method of claim 56, wherein the chemical product is a monoterpene or monoterpenoid, a sesquiterepene or sesquiterpenoid, or a diterpene or diterpenoid.

58. The method of claim 56 or 57, wherein the microbial strain is a bacterial strain, such as E. coli, that expresses one or more additional copies of one or more MEP pathway enzyme.

59. The method of claim 56, wherein the microbial strain is a yeast strain that expresses one or more additional copies of one or more MVA pathway enzymes.

60. The method of claim 58 or 59, wherein the microbial strain expresses one or more of a geranyl diphosphate synthase (GPS), a geranylgeranyl diphosphate synthase (GGPS), a farnsesyl diphosphate synthase (FPS), or a farnesyl geranyl diphosphate synthase (FGPPS), and which is optionally heterologous.

61. The method of claim 60, wherein the microbial strain expresses one or more terpenoid synthase enzymes.

62. The method of claim 61, wherein the microbial strain expresses one or more P450 enzymes synthesizing the chemical product, and the P450 enzyme is optionally heterologous.

63. The method of any one of claims 56 to 62, wherein the product is an oil.

64. The method of claim 63, wherein the product comprises one or more components of an essential oil selected from ylang-ylang, tuberose, rose, neroli, mimosa, jasmine, champaca, bergamot, grapefruit, lemon, lime, orange, sandalwood, petitgrain, patchouli, pepper, costus, cypress, agarwood, jatamansi, kapoor kachri, saffron, ambrette, clary sage, orris, vanilla, and vetiver.

65. The method of claim 64, wherein the product comprises a terpene or terpenoid selected from (−)-khusimone, (−)-limonene, (−)-methyl-(1R, 2R, 5S)-khusimal, (−)-methyl-(1R, 2S, 5S)-khusimal, (−)-rotundone, (+)-aromadendrene, (+)-khusimone, (+)-limonene, (+)-nootkatone, (1R, 2R, 5S)-khusimal, (1R, 2S, 5S)-khusimal, 1,4-cineole, 10-epi-gamma-eudesmol, 4-carvomenthenol, 4-terpineol, abietadiene, abietic acid, acetyl beta-caryophyllene, agarofuran, agarospirol, alpha pinene, alpha-bisabolol, alpha-cedrene, alpha-copaene, alpha-copaene-11-ol, alpha-damascone, alpha-eudesmol, alpha-funebrene, alpha-guaiene, alpha-gurjunene, alpha-humulene, alpha-santalene, alpha-santalol, alpha-selinene, alpha-sinensal, alpha-terpineol, alpha-terpinolene, alpha-vetivone, ambroxan/ambrein, amorphadiene, aristolene, aromadendrene, artemisinic acid, asiatic acid, astaxanthin, atisane, bergamotene, beta pinene, beta-bisabolene, beta-bisabolol, beta-carotene, beta-caryophyllene, beta-damascone, beta-eudesmol, beta-guaiene, beta-santalene, beta-santalol, beta-sinensal, beta-thujone, beta-vetivenene, beta-vetivone, beta-ylangene, bisabolol, boswelic acid, camphene, camphor, carvacrol, carveol, carvone, caryophyllene oxide, cedrenes, celastrol, cembrene, ceroplastol, cineol, cis-abienol, citral, citronellal, citronellol, copalol, cubebol, cucurbitane, cyperene, cyperene epoxide, cyperotundone, damascenone, dehydrofukinone, delta-cadinene, delta-damascone, delta-guaiene, delta-selinene, dihydro agarofuran, E-alpha-bisabolene, E-gamma-bisabolene, eleutherobin, epi-b-santalol, epi-zizaene, epi-zizaenone, eugenol, evopimaradene, farnescene, farnesol, fenchone, forskolin, gamma-bisabolol, gamma-cadinene, gamma-eudesmol, gamma-gurjunene, gamma-humulene, gamma-muurolene, gamma-terpinene, gascardic acid, geraniol, geranylgeraniol, germacrene D, glycyrrhizin, guaiol, guaiene, haslene, ionones, iripallidal, irones, isoborneol, isopemaradiene, isoprene, iso-velencenol, jinkohols, karanone, kaurene, kessane, khusimene, khusimol, labdenediol, ledene, ledol, levopimaradiene, levopimaric acid, linalool, linalool oxide, longifolenaldehyde, longipinene, L-rose oxide, lupeol, madeccasic acid, menthol, menthone, methyl vetivenate, mogrosides, muurolenes, myrcene, nerolidol, nootkatene, nootkatol, nootkatone, ocimenes, ophiobolin A, patchouli alcohol, pinene, piperitone, pogostol, prenol, protopanaxadiol, protopanaxatriol, pulegone, R-(−)-carvone, rotundone, S-(+)-carvone, sabinene, sabinene hydrate, santalals, santalenes, santalols, sclarene, sclareol, selina-3, 7(11)-diene, selinadiene, spathulenol, steviol, steviol glycosides, sulcatone, tagetone, taxadiene, thymol, ursolic acid, valencene, valeranone, verbenone, vetiverol, vetiverone, vetiveryl acetate, viridiflorol, Z, E-alpha-bergamotol, Z-alpha-bisabolene, zeaxanthin, Z-gamma-bisabolene, zizaene, zizenone, and Z-lanceol.

66. The method of any one of claims 55 to 65, wherein a microbial cell population expresses heterologous gene encoding one or more of a uridine diphosphate dependent glycosyltransferase enzyme (UGT), a methyltransferase enzyme, a acetyltransferase enzyme, or a benzoyl transferase enzyme.

67. The method of claim 66, wherein the chemical product is selected from a terpenoid glycoside, flavonoid glycoside, cannabinoid glycoside, polyketide glycoside, stilbenoid glycoside, and polyphenol glycoside.

68. The method of claim 66 or 67, wherein the cultured cell catalyzed enzymatic conversion of a fed substrate.

69. The method of claim 68, wherein the chemical product is a terpenoid glycoside, which is optionally a steviol glycoside or mogrol glycoside.

70. The method of claim 68 or 69, wherein the fed substrate is plant extract, and which is optionally a stevia leaf extract or fraction thereof, or a monkfruit extract or fraction thereof.

71. The method of any one of claims 1 to 70, wherein the bioreactor is a stirred tank reactor or a bubble column bioreactor.

72. A fermentation system for conducting the method of any one of claims 1 to 71.

73. A fermentation system comprising a bioreactor vessel containing growth media and a microbial biomass, the system further comprising: a filtration membrane removing permeate from the bioreactor vessel in situ while retaining biomass; a feeding vessel comprising fresh broth fluidly connected to the bioreactor vessel; and a vessel for collecting filtrated broth fluidly connected to the filtration membrane.

74. A fermentation system comprising a bioreactor vessel containing growth media and a microbial biomass, the system further comprising: a cross filtration module (CFM) fluidly connected to the bioreactor vessel, the CFM comprising a membrane removing permeate from the growth media ex situ while retaining biomass; a feeding vessel comprising fresh broth fluidly connected to the bioreactor vessel; and a vessel fluidly connected to the CFM for collecting filtrated broth, while retentate comprising biomass is recirculated to the bioreactor via a recirculation line.

75. The fermentation system of claim 73 or 74, further comprising a feeding line feeding fresh broth from the feeding vessel and a peristaltic pump operably connected to the feeding line.

76. The fermentation system of claim 75, wherein the feeding line further comprises a feeding line valve.

77. The fermentation system of claim 75, further comprising a line fluidly connected to the bioreactor vessel and the CFM, and comprising a peristaltic pump removing media and biomass from the bioreactor.

78. The fermentation system of any one of claims 74 to 77, wherein the bioreactor vessel is a stirred tank reactor or bubble column reactor.

79. The fermentation system of any one of claims 74 to 78, wherein the bioreactor system further comprises operably connected to the bioreactor vessel: pH sensor, air supply with air filter, dissolved oxygen sensor, and temperature sensor.

80. A method for making a chemical product by microbial fermentation, comprising:
providing a cell culture producing the chemical product from carbon substrate or by enzymatic bioconversion of a fed substrate, each optionally through one or more heterologously expressed enzymes;
culturing the cell in a fermentation media in a bioreactor under conditions suitable for producing the chemical product in the presence of a defined media comprising an amino acid mix and/or pyruvate; and
recovering the chemical product from the fermentation media or an organic overlayer.

81. The method of claim 80, wherein the process is fed-batch, semi-continuous, or continuous.

82. The method of claim 80 or 81, wherein yeast extract is partly or completely replaced with the defined media.

83. The method of any one of claims 80 to 82, wherein the defined media comprises supplementation with pyruvate.

84. The method of any one of claims 80 to 83, comprising a growth phase and a production phase.

85. The method of claim 84, wherein the growth phase and/or the production phase comprises feeding a nitrogen source and a carbon source.

86. The method of any one of claims 80 to 85, the defined media comprises at least amino acids that are synthesized from pyruvate.

87. The method of claim 86, wherein the amino acids comprise L-Ala, L-Val, and L-Leu.

88. The method of claim 87, wherein the amino acid mix comprises, consists essentially of, or consists of L-Ala, L-Glu, Gly, L-Leu, L-Val, and L-Ile.

89. The method of any one of claims 80 to 88, wherein the culturing is conducted without yeast extract.
